(19) United States
(12) Patent Application Publication
(10) Pub. No.: US 2017/0240522 A1
(43) Pub. Date: Aug. 24, 2017

(54) BENZOTHIOPHENE, BENZYLOXYBENZYLIDENE AND INDOLINE DERIVATIVES USEFUL FOR THE TREATMENT OF TUBERCULOSIS

(71) Applicant: École Polytechnique Fédérale de Lausanne (EPFL), Lausanne (CH)

(72) Inventors: Jan RYBNIKER, Lausanne (CH); Stewart COLE, Lausanne (CH); György KERI, Budapest (HU); László ORFI, Budapest (HU); János PATO, Budapest (HU); István SZABADKAI, Budapest (HU); Péter BANHEGYI, Budapest (HU); Zoltán GREFF, Budapest (HU); Péter MARKO, Veresegyház (HU)

(21) Appl. No.: 15/510,872

(22) PCT Filed: Sep. 15, 2015
(86) PCT No.: PCT/EP15/71110

§ 371 Date: Mar. 13, 2017

(30) Foreign Application Priority Data
Sep. 15, 2014 (EP) 14184847.3

(51) Int. Cl. C07D 333/68; C07C 335/40; G01N 33/50; C07D 209/40; C07D 401/14; C07D 295/194; C07C 337/04; C07D 403/06

(52) U.S. Cl. CPC C07D 333/68; C07C 337/04; C07C 335/40; C07D 403/06; C07D 209/40; C07D 401/14; C07D 295/194; G01N 33/5038; G01N 2500/10

(57) ABSTRACT

The present invention relates to benzothiophene, benzyloxybenzylidene and indoline-2-one derivatives and the use of said derivatives in the treatment and/or prevention of tuberculosis.

### TECHNICAL FIELD

The present invention relates to benzothiophene, benzyloxybenzylidene and indoline-2-one derivatives and the use of said derivatives in the treatment and/or prevention of tuberculosis.

### BACKGROUND OF THE INVENTION

Tuberculosis, resulting from infection with Mycobacterium tuberculosis (Mtb), is a serious global health problem accounting for 1.4 million deaths in 2011. A major reason for the high morbidity and mortality caused by Mtb is the long duration of therapy and increasing multidrug-resistance.
Mtb harbors essential protein export systems like the general secretory pathway (Sec) and the twin-arginine pathway (Tat) which process the majority of the mycobacterial secretome. Five specialized ESX or type VII secretion systems are dedicated to the secretion of protein subsets such as virulence determinants. Among these, the ESX-1 system represents an important virulence protein secretion machinery. The ESX-1 secretion apparatus is a complex multi-component translocation system composed of several transmembrane proteins, ATPases and essential accessory proteins which ensure transport of the protein substrates across the mycobacterial membrane.
Additionally, there are several key regulatory proteins that co-regulate ESX-1 secretion. Experiments performed on a wide range of ESX-1 mutants have demonstrated that ESX-1-dependent substrates are essential for host-cell invasion, intracellular replication and inhibition of phagosome maturation. The best understood ESX-1 substrate, EsxA, a 6 kDa protein, is capable of lysing cell membranes leading to cytosolic escape and subsequent dissemination of Mtb.
After screening compounds for growth inhibition of Mtb in conventional drug screens in vitro, some progress has been made towards the implementation of bioactive molecules with new mechanisms of action in clinical trials. However, alternative effective agents are needed to combat Mycobacterium tuberculosis and multi-drug-resistant Mycobacterium tuberculosis virulence with efficacy and fewer adverse reactions.

### SUMMARY OF THE INVENTION

The present invention provides an inhibitor of mycobacterium virulence of general formula (I)

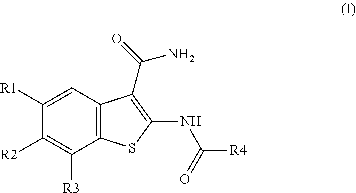

wherein:
R1 is selected from the group consisting of H, halogen, amine;
R2 is selected from the group consisting of H, —OH, substituted alkoxy, —O(CH₂)ₙ—NH₂ with n=2 to 5, acyloxy;
R3 is selected from the group consisting of H, halogen, C₁-C₆ alkyl;
R4 is selected from the group consisting of amine, substituted amine, C₃-C₈ cycloalkyl, substituted benzene,
or general formula (IIA)

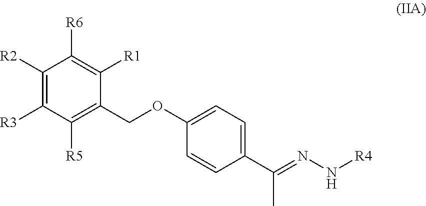

wherein:
R1 is selected from the group consisting of H, halogen, alkoxy;
R2 is selected from the group consisting of H, halogen, nitrogen dioxide, —CF₃, —CO—ORₐ wherein Rₐ is C₁-C₆ alkyl, —SO₂—R_{b} wherein R_{b} is phenyl;
R3 is selected from the group consisting of H, halogen, nitrogen dioxide;
R4 is selected from the group consisting of H, —C(S)—S—R′, —C(S)—NH—R′, —C(S)—NR_{c}—R′ wherein R′ is H, C₁-C₆ alkyl, C₁-C₆ alkene or substituted benzene and R_{c} is substituted C₁-C₆ alkyl;
R5 is selected from the group consisting of H, halogen, cyano group;
R6 is selected from the group consisting of H, halogen,
or, general formula (III)

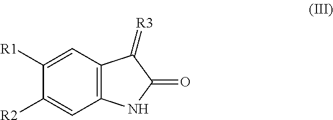

wherein:
R1 is selected from the group consisting of H, halogen, nitrogen dioxide, carboxy, alkoxy, heteroaryl;
R2 is selected from the group consisting of H, halogen;
R3 is selected from the group consisting of C₁-C₆ alkyl heteroaryl, ═N—NH—R″ wherein R″ is substituted aryl;
and/or pharmaceutically acceptable salts thereof.
The invention also provides said inhibitors for use as a medicament, their use in the treatment and/or prevention of tuberculosis as well as pharmaceutical compositions comprising said inhibitors.
A further object of the present invention is to provide a screening method for identifying inhibitors of mycobacterium virulence, said method comprising

a) infecting eukaryotic cells and/or macrophages with wild-type Mtb-Erdman strain at high multiplicities of infection (MOI),

b) contacting said infected eukaryotic cells and/or infected macrophages with an inhibitor to be screened,

c) quantifying metabolic activity in said eukaryotic cells and/or macrophages,

wherein said inhibitor fulfills the following criteria:

i) protects said eukaryotic cells and/or macrophages from Mycobacterium tuberculosis (Mtb)-induced cell death during and after the exposure to the said inhibitor,

ii) does not influence Mtb growth, and

iii) either inhibits the histidine kinase MprB in Mtb or affects metal ion homeostasis in Mtb.

### BRIEF DESCRIPTION OF THE FIGURES

FIG. 1 shows principle of the fibroblast based HTS for identification of protein secretion inhibitors. (A) Pipetting and incubation scheme of the FSA. For drug screens, compounds were added to empty 384 well plates followed by addition of fibroblasts. (B) Well defined ESX-1 mutants are deficient in killing fibroblasts (mean values and standard deviation (±SD)). (C) Antimycobacterium compounds with intracellular activity protect fibroblasts from Mtb-induced cytotoxicity (Compound concentration 10 μM, ±SD). (D) Plate-layout for HTS and identification scheme for putative protein secretion inhibitors.

FIG. 1(E, F) shows that eukaryotic kinase inhibitors are not active in the FSA; the Z′-factor of the FSA is >0.5. (E) A selection of kinase inhibitors which are known to reduce the intracellular mycobacterium load in macrophages were screened in the FSA and in the REMA. None of the compounds protects lung-fibroblasts from Mtb-induced cell lysis (±SD). (F) Z′-factor of the FSA was determined using 384 well plates and the controls DMSO (black data points) and rifampicin (red data points). Statistical calculations were done as described recently (Zhang et al., 1999). The Z′-factor of the displayed experiment is 0.626 indicating a high quality assay.

FIG. 2(A, B, C, D) shows outcome of the primary and confirmatory screen. (A) Hit rate of FSA and REMA in primary and confirmatory screens. (B) Potency of 55 FSA hit compounds (5 μM) in comparison to the controls rifampicin (5 μg/ml) and DMSO. Core structures of the three most abundant scaffolds. (C) BTP15 and BBH7 protect fibroblasts from Mtb-induced killing in a dose dependent manner. (D) BTP15 has no influence on GFP expression by Mtb indicating that the compound is not bactericidal in fibroblasts (Compound concentration 5 μM, ±SD) whereas BBH7 reduces the GFP-signal comparable to rifampicin-treated fibroblasts.

FIG. 2(E, F, G) shows molecular structures of BBH7 and BTP15, both compounds are not growth inhibitory in broth (E) Molecular structures of BTP15 and BBH7. (F) Growth curves of Mtb-Erdman treated with 25 μM of BTP15 or BBH7. The compounds are not growth inhibitory at concentrations which are 10× (BBH7) or 20× (BTP15) higher than the IC50 determined in the FSA. Rifampicin was used as a control at 5 μg/ml. Representative example of three individual experiments. (G) IC99 of BBH7 and BTP15 against a panel of mycobacterial and non-mycobacterial pathogens. Rifampicin (RIF) was used as a control.

FIG. 3 shows that BTP15 and BBH7 affect EsxA secretion of Mtb. Bacteria were exposed to different concentrations of compound. After four days EsxA, Ag85 and the cell lysis control GroEL were detected by western blot in the culture filtrate (CF) and culture lysate (CL).

FIG. 4 shows that BTP15 is a kinase inhibitor that deregulates genes of the MprAB regulon. (A) qRT-PCR of BTP15 treated samples. The compound leads to downregulation (>1.5 fold) of DosR/MprAB associated genes and upregulation (>2 fold) of espA (±SD). (B) Transcriptional levels of three two-component regulatory genes followed by qRT-PCR over three different time-points after treatment with two different concentrations of BTP15. The compound leads to downregulation of mprA after 24 and 48 hours of treatment (±SD). (C) Coomassie blue stained SDS-PAGE of affinity purified MprB and autophosphorylation of MprB after incubation with [γ-32P]ATP detected by autoradiography in a second SDS-PAGE with similar loading. (D) 25 μM of MprB were treated with different concentrations of BTP15 and incorporation of 32P was quantified by scintillation counting. BTP15 leads to a dose-dependent inhibition of autophosphorylation. Non-hydrolysable AMP-PNP was used as a control at 1 and 10 mM (±SD).

FIG. 5(A, B, C, D) shows that BBH7 induces several P-type-ATPases and alters outer membrane permeability. (A) A selection of up- and down-regulated genes upon exposure with BBH7 (5 μM). (B) BBH7 treatment (10 μM) leads to increased EtBr uptake indicating altered outer membrane permeability. Representative example of three individual experiments. (C) Addition of zinc strongly induces EsxA secretion in a dose dependent manner. The Tat secretion substrate Ag85 is not affected by this treatment. Band intensity of EsxA in the CF was quantified in the lower panel. (CF: culture filtrate, CL: culture lysate; representative example of three individual experiments). (D) BBH7 and BTP15 (10 μM) have no impact on ATP-levels of Mtb, the ATP-synthase inhibitor Bedaquiline (BDQ, 60 ng/ml) was used as a control. Relative light units (RLU) were adjusted to OD values (±SD).

FIG. 5(E, F, G) shows gene categories of BBH7-deregulated genes, confirmation by qRT-PCR and western blot targeting EsxA after treatment with cell wall inhibitors. (E) Distribution of 144 BBH7 differentially regulated genes in different gene categories as determined by RNA-seq. (F) Transcription levels of differentially regulated genes upon BBH7 treatment determined by qRT-PCR. Data are derived from three biological replicates (±SD). (G) Western blot targeting EsxA in the culture filtrate of Mtb-treated with different cell wall biosynthesis inhibitors. EsxA and GroEL (lysis control) were detected in the culture filtrate of Mtb-Erdman treated with different cell wall biosynthesis inhibitors as well as BBH7 and BTP15. None of the well described drugs have an impact on EsxA secretion. INH: Isoniazid, TAC: thioacetazone, ETH: ethionamide, EMB: ethambutol.

FIG. 6 shows BTP15 and BBH7 that promote phago-lysosomal fusion and reduce bacterial load in activated THP-1 macrophages. (A/B) Confocal microscopy of infected THP-1 macrophages after treatment with the two compounds (10 μM) or vehicle (DMSO). After 7 days, acidic compartments were stained with Lysotracker Red and co-localization of Mtb-GFP with these compartments was quantified (Scale bar: 20 μm). Both compounds promote phagolysosomal fusion to a higher rate than DMSO-treated bacteria. P-values≦0.001=***; ≦0.01=** (±SD). (C) Survival of activated THP-1 macrophages was quantified as performed with MRC-5 lung fibroblasts. Both compounds (10 μM) protect the cells from Mtb-induced cytotoxicity. (D/E) Mtb-GFP was quantified inside activated THP-1 cells after treatment with BBH7 and BTP15 (10 μM) as described in the methods section. Both compounds significantly reduce the intracellular bacterial load. For BTP15 this stands in contrast to treatment of infected fibroblasts where intracellular replication is not affected (FIG. 2D) (Scale bar: 100 μm).

FIG. 7 shows model for zinc-induced EsxA secretion (A) and implications for BBH7 function (B). (A) Upon phagocytosis of Mtb macrophages recruit heavy metal transporting ATPases like ATP7A to the phagosomal membrane leading to the intraphagosomal accumulation of toxic amounts of copper and zinc. This rapidly triggers a mycobacterial response involving the upregulation of P-type ATPases (CtpC/CtpG) and metal-chelating proteins dedicated to the clearance of intracellular copper and zinc. In addition, elevated zinc concentrations induce the secretion of EsxA subsequently leading to phagosomal damage and ion-efflux, thus providing a second line of defense against host driven heavy metal intoxication. (B) Treatment with BBH7 alters mycobacterial outer membrane permeability leading to transcriptional signs of copper and zinc stress. CtpC and CtpG will promote heavy metal efflux into the phagosomal vacuole. In parallel, the ESX-1 translocating ATPases EccCa1 and EccCb1 are upregulated, however, EsxA secretion is blocked by an unknown mechanism probably leading to phagosomal integrity and a vicious circle of further accumulation of heavy metals in the phagosome and poisoning of Mtb.

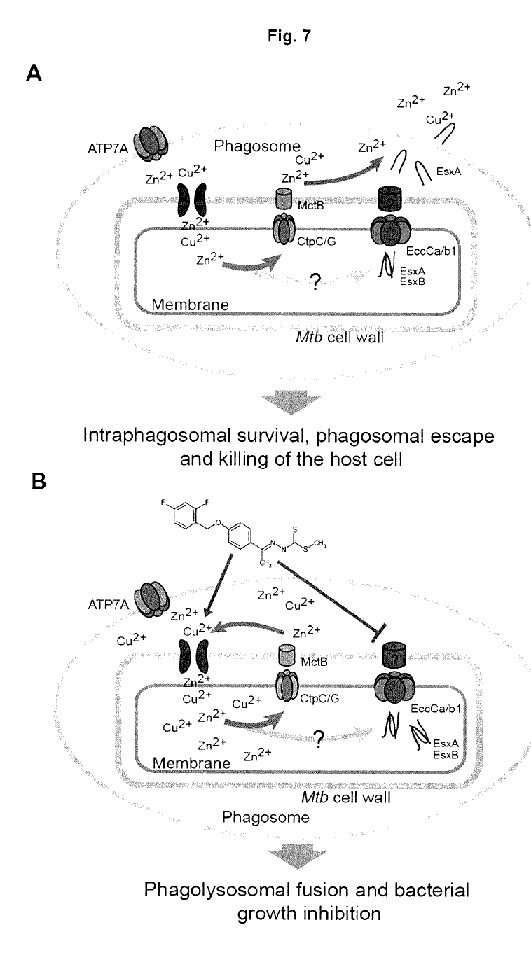

### DETAILED DESCRIPTION OF THE INVENTION

Mycobacterium tuberculosis (Mtb) depends on protein secretion systems like ESX-1 for intracellular survival and virulence. The ESX-1 secretion apparatus is a complex multi-component translocation system composed of several transmembrane proteins, ATPases and essential accessory proteins which ensure transport of the protein substrates across the mycobacterium membrane. Additionally, there are several key regulatory proteins that co-regulate ESX-1 secretion. In particular, the ESX-1 substrate EsxA, a 6 kDa protein, is capable of lysing cell membranes leading to cytosolic escape and subsequent dissemination of Mtb. EsxA is a major virulence determinant that causes tissue damage and necrosis, thereby promoting pathogen spread and dissemination.
The Applicant developed a fibroblast survival assay (FSA) that exploits this phenotype by selecting for compounds that protect host cells from Mtb-induced lysis without being bactericidal in vitro. Several chemical compounds were identified for their ability to block Mycobacterium tuberculosis (Mtb) virulence. Hit compounds identified in high-throughput screen blocked secretion of EsxA thus promoting phagosome maturation and substantially reducing bacterial burden in activated macrophages.
As used herein, the following definitions are supplied in order to facilitate the understanding of the present invention.
As used herein, the term “comprise” is generally used in the sense of include, that is to say permitting the presence of one or more features or components.
As used herein, the singular form “a”, “an” and “the” include plural references unless the context clearly dictates otherwise.
As used herein, the term “mycobacterium virulence” refers to the bacterial genes and/or proteins of the ESX-1 protein secretion system that are essential for the bacteria to trigger tuberculosis infection.
As used herein, the term “inhibitor” refers to compounds that block or partially block directly or indirectly the activity of proteins, and/or the secretion of proteins, and/or deregulate genes involved in mycobacterium virulence without affecting mycobacterial growth.

In one aspect, the present invention provides a compound of general formula (I)

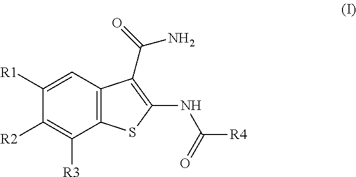

wherein:
R1 is selected from the group consisting of H, halogen, amine;
R2 is selected from the group consisting of H, —OH, substituted alkoxy, —O(CH₂)ₙ—NH₂ with n=2 to 5, acyloxy;
R3 is selected from the group consisting of H, halogen, C₁-C₆ alkyl;
R4 is selected from the group consisting of amine, substituted amine, C₃-C₈ cycloalkyl, substituted benzene,
or general formula (IIA)

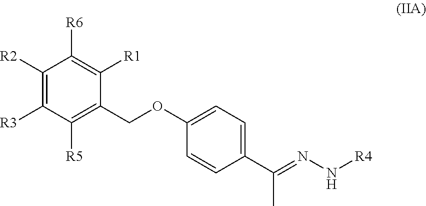

wherein:
R1 is selected from the group consisting of H, halogen, alkoxy;
R2 is selected from the group consisting of H, halogen, nitrogen dioxide, —CF₃, —CO—ORₐ wherein Rₐ is C₁-C₆ alkyl, —SO₂—R_{b} wherein R_{b} is phenyl;
R3 is selected from the group consisting of H, halogen, nitrogen dioxide;
R4 is selected from the group consisting of H, —C(S)—S—R′, —C(S)—NH—R′, —C(S)—NR_{c}—R′ wherein R′ is H, C₁-C₆ alkyl, C₁-C₆ alkene or substituted benzene and R_{c} is substituted C₁-C₆ alkyl;
R5 is selected from the group consisting of H, halogen, cyano group;
R6 is selected from the group consisting of H, halogen,
or, general formula (III)

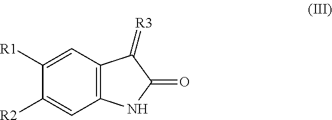

wherein:
R1 is selected from the group consisting of H, halogen, nitrogen dioxide, carboxy, alkoxy, heteroaryl;
R2 is selected from the group consisting of H, halogen;
R3 is selected from the group consisting of C₁-C₆ alkyl heteroaryl, ═N—NH—R″ wherein R″ is substituted aryl;
and/or pharmaceutically acceptable salts thereof.
Preferably, the compound of general formula (I), (IIA) or (III) is an inhibitor of mycobacterium virulence.
In another aspect, the present invention relates to a compound of general formula (I)

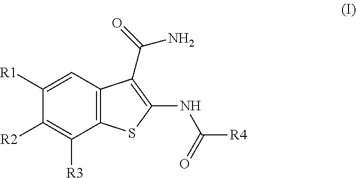

wherein:
R1 is selected from the group consisting of H, halogen, amine;
R2 is selected from the group consisting of H, —OH, substituted alkoxy, —O(CH₂)ₙ—NH₂ with n=2 to 5, acyloxy;
R3 is selected from the group consisting of H, halogen, C₁-C₆ alkyl;
R4 is selected from the group consisting of amine, substituted amine, C₃-C₈ cycloalkyl, substituted benzene,
or general formula (II)

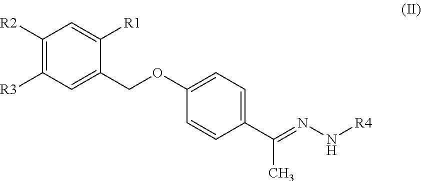

wherein:
R1 is selected from the group consisting of H, halogen, alkoxy;
R2 is selected from the group consisting of H, halogen, nitrogen dioxide;
R3 is selected from the group consisting of H, halogen, nitrogen dioxide;
R4 is selected from the group consisting of —C(S)—S—R′, —C(S)—N—R′ wherein R′ is C₁-C₆ alkyl, or substituted benzene,
or, general formula (III)

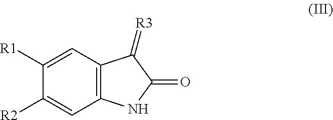

wherein:
R1 is selected from the group consisting of H, halogen, nitrogen dioxide, carboxy, alkoxy, heteroaryl;
R2 is selected from the group consisting of H, halogen;
R3 is selected from the group consisting of C₁-C₆ alkyl heteroaryl, ═N—NH—R″ wherein R″ is substituted aryl;
and/or pharmaceutically acceptable salts thereof.
Preferably, the compound of general formula (I), (II) or (III) is an inhibitor of mycobacterium virulence.
Thus, the present invention relates to an inhibitor of mycobacterium virulence of general formula (I)

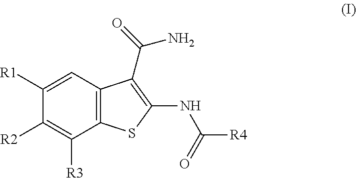

wherein:
R1 is selected from the group consisting of H, halogen, amine;
R2 is selected from the group consisting of H, —OH, substituted alkoxy, —O(CH₂)ₙ—NH₂ with n=2 to 5, acyloxy;
R3 is selected from the group consisting of H, halogen, C₁-C₆ alkyl;
R4 is selected from the group consisting of amine, substituted amine, C₃-C₈ cycloalkyl, substituted benzene,
or general formula (II)

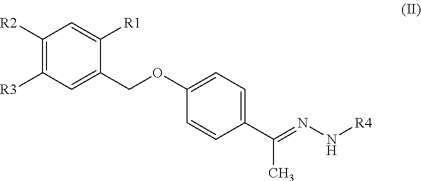

wherein:
R1 is selected from the group consisting of H, halogen, alkoxy;
R2 is selected from the group consisting of H, halogen, nitrogen dioxide;
R3 is selected from the group consisting of H, halogen, nitrogen dioxide;
R4 is selected from the group consisting of —C(S)—S—R′, —C(S)—N—R′ wherein R′ is C₁-C₆ alkyl, or substituted benzene,
or, general formula (III)

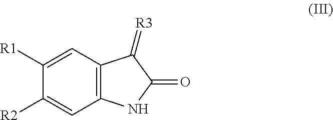

wherein:
R1 is selected from the group consisting of H, halogen, nitrogen dioxide, carboxy, alkoxy, heteroaryl;
R2 is selected from the group consisting of H, halogen;
R3 is selected from the group consisting of C₁-C₆ alkyl heteroaryl, ═N—NH—R″ wherein R″ is substituted aryl;
and/or pharmaceutically acceptable salts thereof.
The present invention further relates to an inhibitor of mycobacterium virulence of general formula (I)

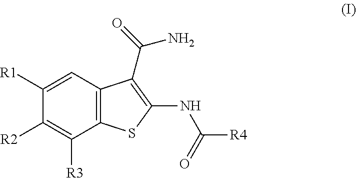

wherein:
R1 is selected from the group consisting of H, halogen, amine;
R2 is selected from the group consisting of H, —OH, substituted alkoxy, —O(CH₂)ₙ—NH₂ with n=2 to 5, acyloxy;
R3 is selected from the group consisting of H, halogen, C₁-C₆ alkyl;
R4 is selected from the group consisting of amine, substituted amine, C₃-C₈ cycloalkyl, substituted benzene, and/or pharmaceutically acceptable salts thereof. Preferably, R4 is a cyclopropane.
The present invention also relates to an inhibitor of mycobacterium virulence of general formula (IIA)

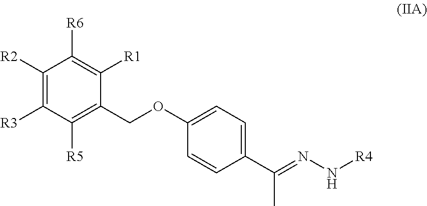

wherein:
R1 is selected from the group consisting of H, halogen, alkoxy;
R2 is selected from the group consisting of H, halogen, nitrogen dioxide, —CF₃, —CO—ORₐ wherein Rₐ is C₁-C₆ alkyl, —SO₂—R_{b} wherein R_{b} is phenyl;
R3 is selected from the group consisting of H, halogen, nitrogen dioxide;
R4 is selected from the group consisting of H, —C(S)—S—R′, —C(S)—NH—R′ wherein R′ is H, C₁-C₆ alkyl, C₁-C₆ alkene or substituted benzene, —C(S)—NR_{c}—R′ wherein R_{c} is substituted C₁-C₆ alkyl and R′ is H, C₁-C₆ alkyl, C₁-C₆ alkene or substituted benzene;
R5 is selected from the group consisting of H, halogen, cyano group;
R6 is selected from the group consisting of H, halogen.
Preferably, R4 is —C(S)—S—CH₃, —C(S)—NH₂, and —C(S)—NH—CH₂—CH═CH₂.
The present invention further relates to an inhibitor of mycobacterium virulence of general formula (II)

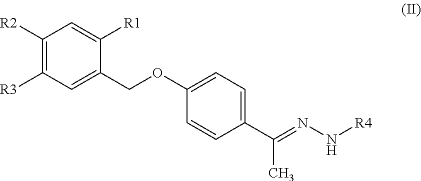

wherein:
R1 is selected from the group consisting of H, halogen, alkoxy;
R2 is selected from the group consisting of H, halogen, nitrogen dioxide;
R3 is selected from the group consisting of H, halogen, nitrogen dioxide;
R4 is selected from the group consisting of —C(S)—S—R′, —C(S)—N—R′ wherein R′ is C₁-C₆ alkyl, or substituted benzene, and/or pharmaceutically acceptable salts thereof. Preferably R4 is —C(═S)—S—CH3.
Alternatively, the present invention relates to an inhibitor of mycobacterial virulence of general formula (III)

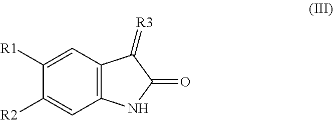

wherein:
R1 is selected from the group consisting of H, halogen, nitrogen dioxide, carboxy, alkoxy, heteroaryl;
R2 is selected from the group consisting of H, halogen;
R3 is selected from the group consisting of C₁-C₆ alkyl heteroaryl, ═N—NH—R″ wherein R″ is substituted aryl, and/or pharmaceutically acceptable salts thereof.
Preferably R3 is

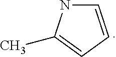

The following paragraphs provide definitions of the various chemical moieties that make up the compounds according to the invention and are intended to apply uniformly throughout the specification and claims unless an otherwise expressly set out definition provides a broader definition.
“Halogen” refers to fluoro, chloro, bromo and iodo atoms.
“C₁-C₆ alkyl” refers to monovalent alkyl groups having 1 to 6 carbon atoms. This term is exemplified by groups such as methyl, ethyl, n-propyl, isopropyl, n-butyl, isobutyl, tert-butyl, n-hexyl and the like.
“C₁-C₆ alkene” refers to an unsaturated hydrocarbon molecule having 1 to 6 carbon atoms that includes a set of carbon-carbon double bonds.
“Amine” refers to —NH₂, —NH—R and —N—RR′ wherein R and R′ are independently H, C₁-C₆-alkyl, benzene, and substituted benzene with one or more group selected from —S—CH3, C₁-C₆ alkyl, —CF₃, halogen (e.g. Cl, F, Br, I), —CH₂—N—(CH₃)₂, —CO—O—CH₃.
“Aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenantrenyl and the like.
“C₁-C₆ alkyl aryl” refers to C₁-C₆-alkyl groups having an aryl substituent, including benzyl, phenethyl and the like.
“Heteroaryl” refers to a monocyclic heteroaromatic, or a bicyclic or a tricyclic fused-ring heteroaromatic group. Particular examples of heteroaromatic groups include optionally substituted, pyrrolyl, pyridyl furyl, thienyl, imidazolyl, oxazolyl, isoxazolyl, thiazolyl, isothiazolyl, pyrazolyl. Preferred heteroaromatic groups is selected from the group comprising pyrrolyl.
“C₁-C₆ alkyl heteroaryl” refers to C₁-C₆-alkyl groups having a heteroaryl substituent. Preferred heteroaryl substituent is selected from the group comprising pyrrolyl and the like.
“Alkoxy” refers to the group —O—R where R includes “C₁-C₆-alkyl”; —O—R—NH₂ where R is “C₁-C₆-alkyl”; —O—R—NH—R′ where R is C₁-C₆-alkyl or C₁-C₆-alkyl hydroxyl and R′ is C₁-C₆-alkyl substituted aryl.
“Acyloxy” refers to the group —OC(O)R where R includes H, “C₁-C₆-alkyl”.
“Nitrogen dioxide” refers to the formula —NO₂.
“Carboxy” refers to the group —C(O)OH.
“C₃-C₈ cycloalkyl” refers to a saturated carbocyclic group of from 3 to 8 carbon atoms having a single ring (e.g., cyclopentyl) or multiple condensed rings. Preferred cycloalkyl include cyclopentyl, and the like.
“Heterocycloalkyl” refers to a C₃-C₈-cycloalkyl group according to the definition above, in which up to 3 carbon atoms are replaced by heteroatoms chosen from the group consisting of O, S, N.
“Cyano group” refers to a carbon atom triple-bonded to a nitrogen atom.
“n=2 to 5” refers to n=2, 3, 4 or 5.
“Substituted or unsubstituted”: Unless otherwise constrained by the definition of the individual substituent, the above set out groups, like “alkyl”, “alkoxy”, “alkenyl”, “alkynyl”, “aryl”, “amine”, “benzene” and “heteroaryl” etc. groups can optionally be substituted with from 1 to 5 substituents selected from the group consisting of “C₁-C₆-alkyl”, “C₂-C₆-alkenyl”, “C₂-C₆-alkynyl”, “cycloalkyl”, “heterocycloalkyl”, “C₁-C₆-alkyl aryl”, “halo C₁-C₆-alkyl aryl”, “C₁-C₆-alkyl heteroaryl”, “C₁-C₆-alkyl cycloalkyl”, “C₁-C₆-alkyl heterocycloalkyl”, “amine”, “amino”, “ammonium”, “acyl”, “acyloxy”, “acylamino”, “aminocarbonyl”, “alkoxycarbonyl”, “ureido”, “carbamate”, “aryl”, “heteroaryl”, “thioalkyl”, “sulfmyl”, “sulfonyl”, “alkoxy”, “sulfanyl”, “halogen”, “haloalkyl”, “carboxy”, “trihalomethyl”, “cyano”, “hydroxyl”, “mercapto”, “nitro”, and the like.

The invention also relates to salts of the inhibitors of mycobacterium virulence of formula (I), (II), (IIA) or (III), chemical modified compounds and derivatives of said inhibitors. Preferably, these salts are pharmaceutically acceptable. According to the present invention, pharmaceutically acceptable salts are produced from acidic inorganic or organic compounds, or alkaline inorganic or organic compounds. As used herein, the phrase “pharmaceutically acceptable salt” refers to a salt that retains the biological effectiveness of the free acids and bases of a specified compound and that is not biologically or otherwise undesirable.
The present invention provides inhibitors of mycobacterium virulence of general formula (I) (Table I) selected from the group comprising:

| Benzothiophene | |
|---|---|
| name | Structure |
| D38979 | 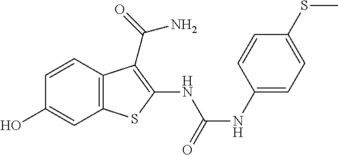 [SMILES: CSc1ccc(NC(=O)Nc2sc3cc(O)ccc3c2C(N)=O)cc1] |
| D39317 | 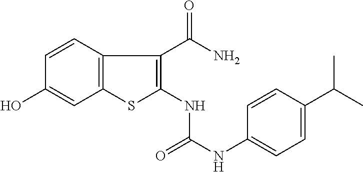 [SMILES: CC(C)c1ccc(NC(=O)Nc2sc3cc(O)ccc3c2C(N)=O)cc1] |
| D39322 | 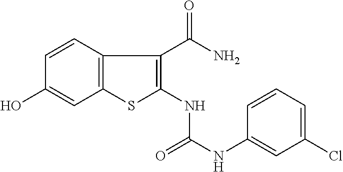 [SMILES: NC(=O)c1c(NC(=O)Nc2cccc(Cl)c2)sc2cc(O)ccc12] |
| D45756 | 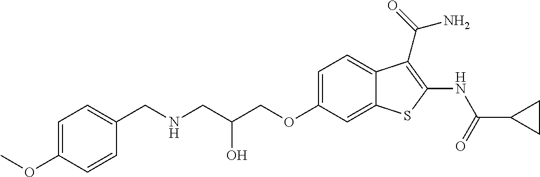 [SMILES: COc1ccc(CNCC(O)COc2ccc3c(C(N)=O)c(NC(=O)C4CC4)sc3c2)cc1] |
| D49399 | 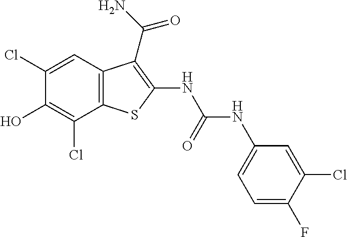 [SMILES: NC(=O)c1c(NC(=O)Nc2ccc(F)c(Cl)c2)sc2c(Cl)c(O)c(Cl)cc12] |
| D51275 | 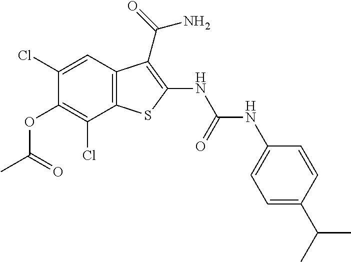 [SMILES: CC(=O)Oc1c(Cl)cc2c(C(N)=O)c(NC(=O)Nc3ccc(C(C)C)cc3)sc2c1Cl] |
| D58298 | 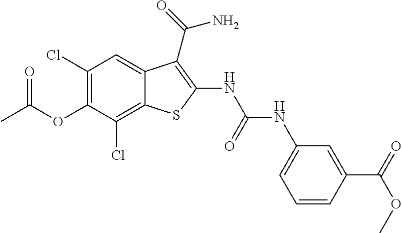 [SMILES: COC(=O)c1cccc(NC(=O)Nc2sc3c(Cl)c(OC(C)=O)c(Cl)cc3c2C(N)=O)c1] |
| D58845 | 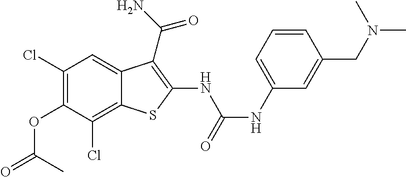 [SMILES: CC(=O)Oc1c(Cl)cc2c(C(N)=O)c(NC(=O)Nc3cccc(CN(C)C)c3)sc2c1Cl] |
| D60166 | 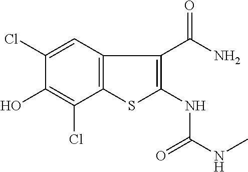 [SMILES: CNC(=O)Nc1sc2c(Cl)c(O)c(Cl)cc2c1C(N)=O] |
| D63134 | 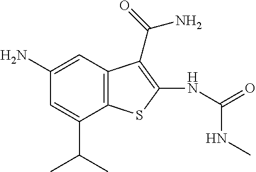 [SMILES: CNC(=O)Nc1sc2c(C(C)C)cc(N)cc2c1C(N)=O] |
| D70865 | 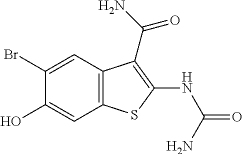 [SMILES: NC(=O)Nc1sc2cc(O)c(Br)cc2c1C(N)=O] |
| D70866 | 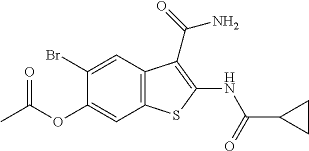 [SMILES: CC(=O)Oc1cc2sc(NC(=O)C3CC3)c(C(N)=O)c2cc1Br] |
| D71014 | 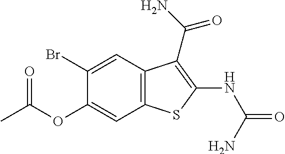 [SMILES: CC(=O)Oc1cc2sc(NC(N)=O)c(C(N)=O)c2cc1Br] |
| D71103 (BTP15) | 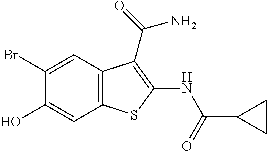 [SMILES: NC(=O)c1c(NC(=O)C2CC2)sc2cc(O)c(Br)cc12] |
| D39321 | 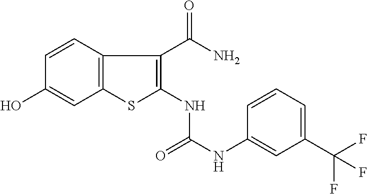 [SMILES: NC(=O)c1c(NC(=O)Nc2cccc(C(F)(F)F)c2)sc2cc(O)ccc12] |

More preferably, the inhibitor of mycobacterium virulence of formula (I) is BTP15 of formula

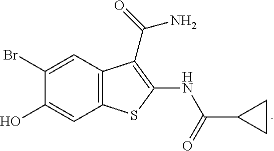 [SMILES: NC(=O)c1c(NC(=O)C2CC2)sc2cc(O)c(Br)cc12]

It has been found that BTP15 is an inhibitor of the histidine kinase MprB that indirectly regulates ESX-1.
Interestingly, the Applicant demonstrates that mycobacterium virulence inhibitors of the invention inhibit the secretion of the major mycobacterium virulence protein EsxA.
Thus, the inhibitors of mycobacterium virulence of general formula I reduce ESX-1-dependent pathogenicity.
Furthermore, BTP15 is a kinase inhibitor that affects EsxA secretion most likely by deregulating the espACD operon. Several transcriptional regulators have been shown to control ESX-1 dependent secretion mainly by binding to this operon which is not part of the ESX-1 region but nonetheless encodes EsxA co321 secreted proteins. An mprAB mutant displayed upregulation of espA and greatly reduced EsxA secretion. Furthermore, MprA coregulates several DosR-regulated genes and SigE.
BTP15 treatment deregulates a similar set of genes and inhibits MprB auto-phosphorylation in vitro. MprAB is clearly associated with virulence since the corresponding mutants show impaired survival in vivo, particularly during the chronic stage of infection. Macrophages infected with a ΔmprAB strain elicit significantly lower levels of tumor necrosis factor alpha (TNF330α) and interleukin 1β (IL-1β) similar to Mtb strains carrying deletions in the espACD operon or the ESX-1 region. However, in contrast to BTP15-treated macrophages, which show reduced intracellular bacterial load, loss of mprAB does not reduce the number of intracellular bacteria in activated macrophages. Low expression levels of dosR, phoP and mprA were revealed by qRT-PCR experiments. Many of the ESX-1 regulatory genes are induced during intracellular infection, thus BTP15 has an extended impact on virulence gene expression inside macrophages and fibroblasts explaining the discrepancy between the intracellular behavior of the ΔmprAB mutant and BTP15-treated bacteria.
The present invention further provides inhibitors of mycobacterium virulence of general formula (IIA) (Table II) selected from the group comprising:

| Code | Structure |
|---|---|
| 14467 | 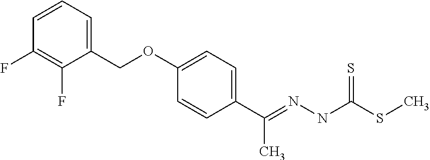 [SMILES: CSC(=S)N/N=C(\C)c1ccc(OCc2cccc(F)c2F)cc1] |
| 14468 | 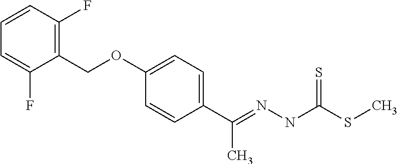 [SMILES: CSC(=S)N/N=C(\C)c1ccc(OCc2c(F)cccc2F)cc1] |
| 14469 | 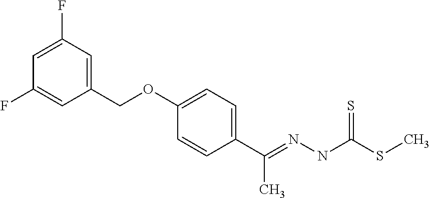 [SMILES: CSC(=S)N/N=C(\C)c1ccc(OCc2cc(F)cc(F)c2)cc1] |
| 14472 | 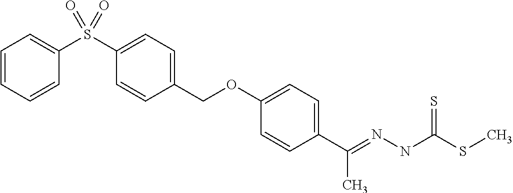 [SMILES: CSC(=S)N/N=C(\C)c1ccc(OCc2ccc(S(=O)(=O)c3ccccc3)cc2)cc1] |
| 14475 | 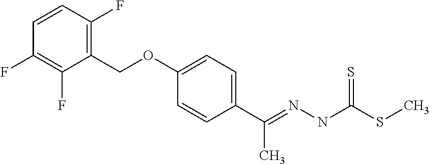 [SMILES: CSC(=S)N/N=C(\C)c1ccc(OCc2c(F)ccc(F)c2F)cc1] |
| 14476 | 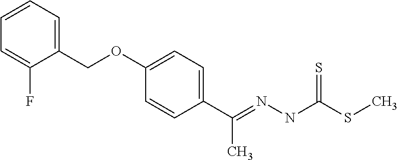 [SMILES: CSC(=S)N/N=C(\C)c1ccc(OCc2ccccc2F)cc1] |
| 14478 | 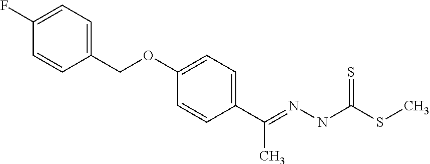 [SMILES: CSC(=S)N/N=C(\C)c1ccc(OCc2ccc(F)cc2)cc1] |
| 14481 | 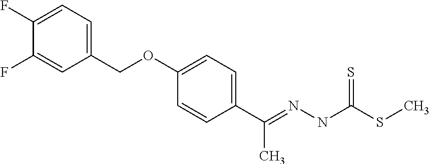 [SMILES: CSC(=S)N/N=C(\C)c1ccc(OCc2ccc(F)c(F)c2)cc1] |
| 14482 | 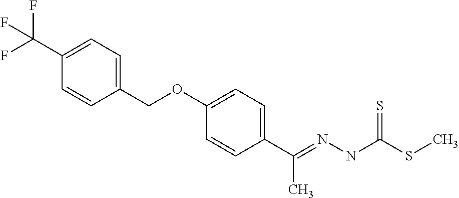 [SMILES: CSC(=S)N/N=C(\C)c1ccc(OCc2ccc(C(F)(F)F)cc2)cc1] |
| 14483 | 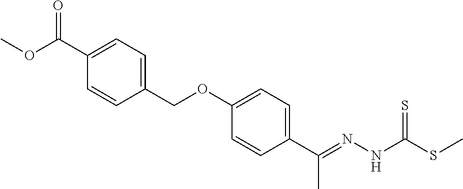 |
| 29985 | 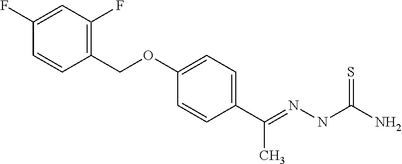 [SMILES: C/C(=N\NC(N)=S)c1ccc(OCc2ccc(F)cc2F)cc1] |
| 30650 | 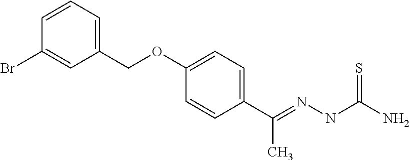 [SMILES: C/C(=N\NC(N)=S)c1ccc(OCc2cccc(Br)c2)cc1] |
| 30651 | 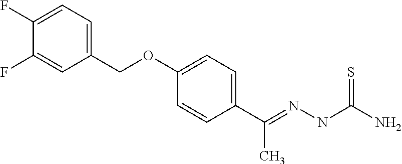 [SMILES: C/C(=N\NC(N)=S)c1ccc(OCc2ccc(F)c(F)c2)cc1] |
| 30652 | 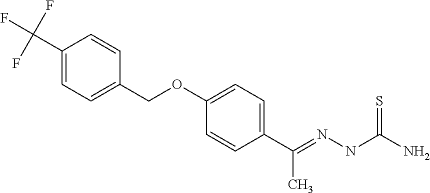 [SMILES: C/C(=N\NC(N)=S)c1ccc(OCc2ccc(C(F)(F)F)cc2)cc1] |
| 30653 | 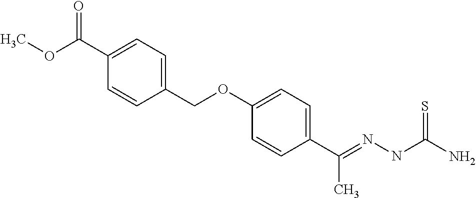 [SMILES: COC(=O)c1ccc(COc2ccc(/C(C)=N/NC(N)=S)cc2)cc1] |
| 30654 | 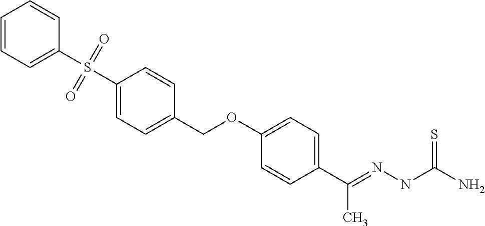 [SMILES: C/C(=N\NC(N)=S)c1ccc(OCc2ccc(S(=O)(=O)c3ccccc3)cc2)cc1] |
| 30655 | 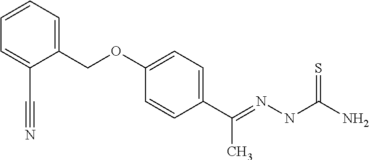 [SMILES: C/C(=N\NC(N)=S)c1ccc(OCc2ccccc2C#N)cc1] |
| 29986 | 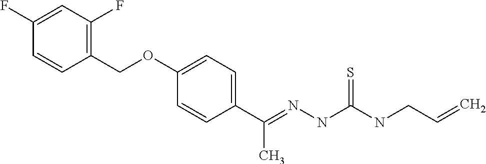 [SMILES: C=CCNC(=S)N/N=C(\C)c1ccc(OCc2ccc(F)cc2F)cc1] |
| 30656 | 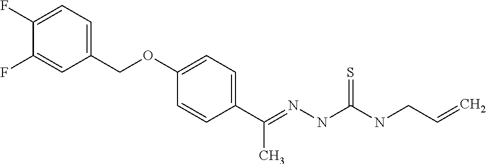 [SMILES: C=CCNC(=S)N/N=C(\C)c1ccc(OCc2ccc(F)c(F)c2)cc1] |
| 30657 | 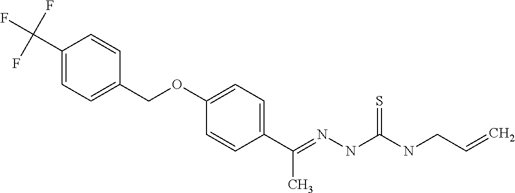 [SMILES: C=CCNC(=S)N/N=C(\C)c1ccc(OCc2ccc(C(F)(F)F)cc2)cc1] |
| 30658 | 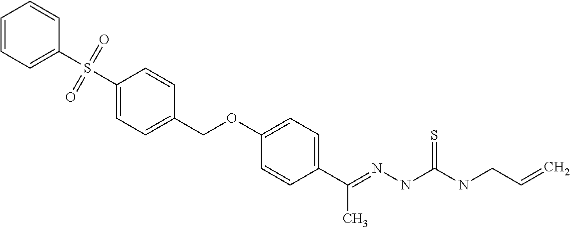 [SMILES: C=CCNC(=S)N/N=C(\C)c1ccc(OCc2ccc(S(=O)(=O)c3ccccc3)cc2)cc1] |
| 30659 | 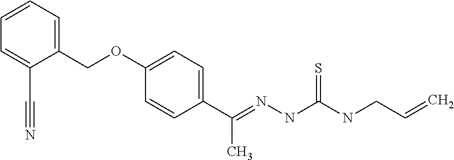 [SMILES: C=CCNC(=S)N/N=C(\C)c1ccc(OCc2ccccc2C#N)cc1] |
| 30660 | 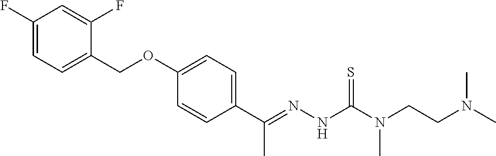 [SMILES: C/C(=N\NC(=S)N(C)CCN(C)C)c1ccc(OCc2ccc(F)cc2F)cc1] |
| 30661 | 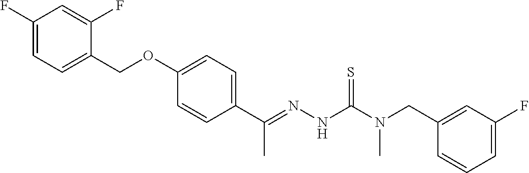 [SMILES: C/C(=N\NC(=S)N(C)Cc1cccc(F)c1)c1ccc(OCc2ccc(F)cc2F)cc1] |
| 14483 | 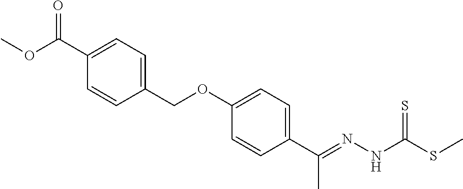 |

The present invention further provides inhibitors of mycobacterium virulence of general formula (II) (Table III) selected from the group comprising:

| Benzyloxybenzylidene | |
|---|---|
| Name | Structure |
| D22670 | 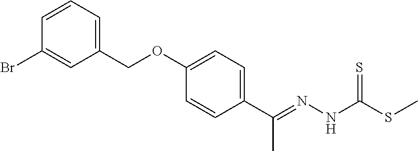 [SMILES: CSC(=S)N/N=C(\C)c1ccc(OCc2cccc(Br)c2)cc1] |
| D22663 | 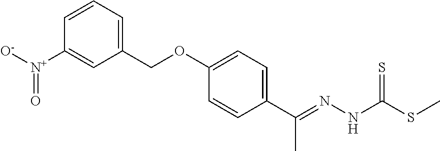 [SMILES: CSC(=S)N/N=C(\C)c1ccc(OCc2cccc([N+](=O)[O-])c2)cc1] |
| D22671 | 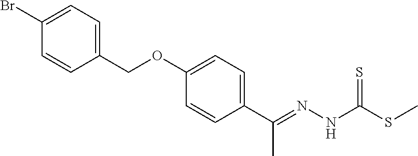 [SMILES: CSC(=S)N/N=C(\C)c1ccc(OCc2ccc(Br)cc2)cc1] |
| D23251 | 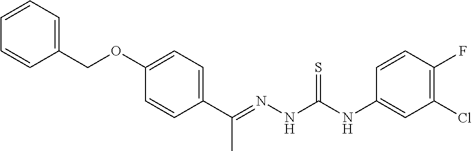 [SMILES: C/C(=N\NC(=S)Nc1ccc(F)c(Cl)c1)c1ccc(OCc2ccccc2)cc1] |
| D22672 | 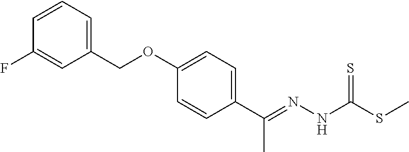 [SMILES: CSC(=S)N/N=C(\C)c1ccc(OCc2cccc(F)c2)cc1] |
| D22668 (BBH7) | 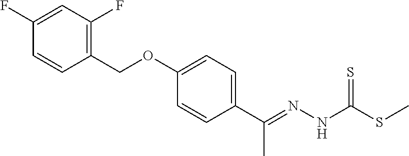 [SMILES: CSC(=S)N/N=C(\C)c1ccc(OCc2ccc(F)cc2F)cc1] |
| D23579 | 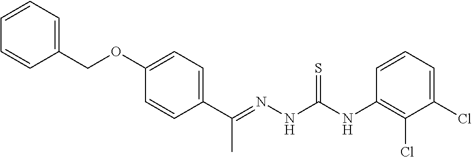 [SMILES: C/C(=N\NC(=S)Nc1cccc(Cl)c1Cl)c1ccc(OCc2ccccc2)cc1] |
| D22647 | 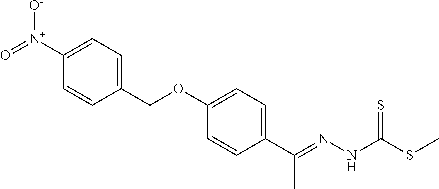 [SMILES: CSC(=S)N/N=C(\C)c1ccc(OCc2ccc([N+](=O)[O-])cc2)cc1] |
| D22646 | 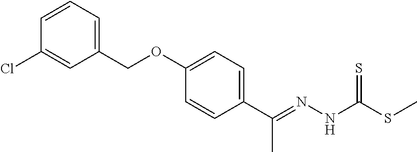 [SMILES: CSC(=S)N/N=C(\C)c1ccc(OCc2cccc(Cl)c2)cc1] |
| D22648 | 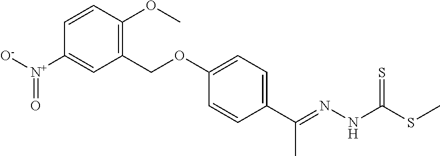 [SMILES: COc1ccc([N+](=O)[O-])cc1COc1ccc(/C(C)=N/NC(=S)SC)cc1] |
| 14463 | 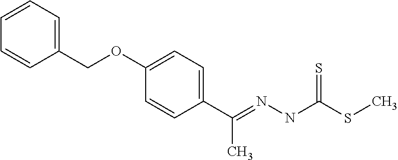 [SMILES: CSC(=S)N/N=C(\C)c1ccc(OCc2ccccc2)cc1] |
| 14466 | 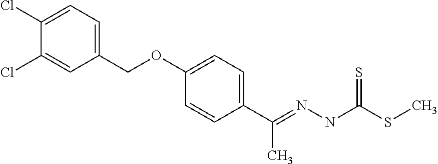 [SMILES: CSC(=S)N/N=C(\C)c1ccc(OCc2ccc(Cl)c(Cl)c2)cc1] |

More preferably, the inhibitor of mycobacterium virulence of formula (II) is BBH7 of formula:

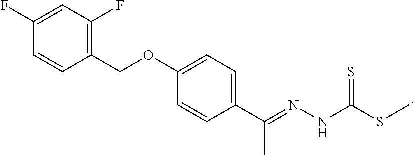 [SMILES: CSC(=S)N/N=C(\C)c1ccc(OCc2ccc(F)cc2F)cc1]

Furthermore, inhibitors of the general formula II are able to disturb bacterial membrane permeability. It has been found that inhibitors of the general formula II comprising BBH7 affects metal ion homeostasis in Mtb and revealed zinc stress as a signal for EsxA secretion.

With BBH7 the Applicant identified a pleiotropic inhibitor of mycobacterial protein secretion. However, the gene expression signature following exposure to BBH7, with strong upregulation of several P-type ATPases as well as altered EtBr uptake in treated bacteria, suggests disturbed export not only for proteins but also for smaller molecules. This makes a common pore structure exclusively dedicated to protein transport through the cell envelope an unlikely target of BBH7. Rather, it is conceivable that this compound has a more general impact on processes involved in cell wall biogenesis leading to misincorporation or disassembly of several translocation-associated structures. In fact, supra-molecular structures dedicated to outer membrane transport are often linked to cell wall components and loss of this interaction blocks protein secretion.
The present invention also provides inhibitors of mycobacterial virulence of general formula (III) (Table IV) selected from the group comprising:

| Indoline- | |
|---|---|
| 2-one | |
| Name | Structure |
| D1364 | 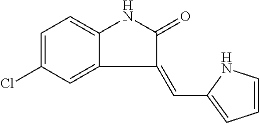 [SMILES: O=C1Nc2ccc(Cl)cc2/C1=C/c1ccc[nH]1] |
| D35964 | 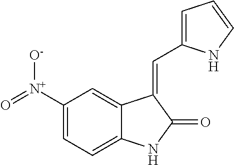 [SMILES: O=C1Nc2ccc([N+](=O)[O-])cc2/C1=C/c1ccc[nH]1] |
| D2886 | 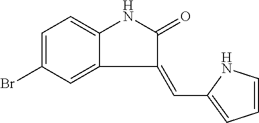 [SMILES: O=C1Nc2ccc(Br)cc2/C1=C/c1ccc[nH]1] |
| D6304 | 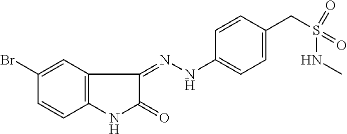 [SMILES: CNS(=O)(=O)Cc1ccc(N/N=C2\C(=O)Nc3ccc(Br)cc32)cc1] |
| D6767 | 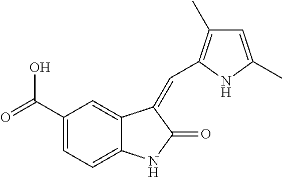 [SMILES: Cc1cc(C)c(/C=C2\C(=O)Nc3ccc(C(=O)O)cc32)[nH]1] |
| D36097 | 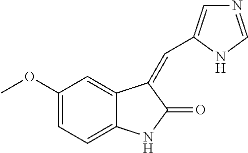 [SMILES: COc1ccc2c(c1)/C(=C/c1cnc[nH]1)C(=O)N2] |
| D51233 | 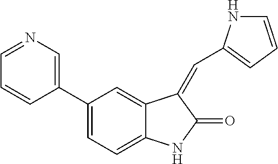 [SMILES: O=C1Nc2ccc(-c3cccnc3)cc2/C1=C/c1ccc[nH]1] |
| D60330 | 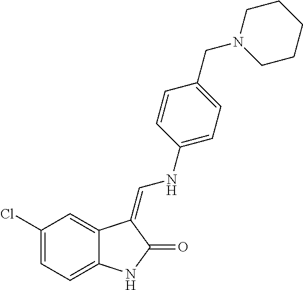 [SMILES: O=C1Nc2ccc(Cl)cc2/C1=C/Nc1ccc(CN2CCCCC2)cc1] |

Mycobacterium is a genus of Actinobacteria, given its own family, the Mycobacteriaceae. The Mycobacterium genus is usually separated into two major groups on the basis of their growth rate. One group includes slow-growing species such as the well-known pathogens Mycobacterium tuberculosis, Mycobacterium bovis and Mycobacterium leprae (ethiological agents of human tuberculosis (TB), bovine tuberculosis (BTB) and leprosy respectively); the other group gathers fast-growing species such as Mycobacterium smegmatis, which in general are opportunistic or non-pathogenic bacteria.
The Mycobacterium tuberculosis complex (MTBC) refers to group of species (M. tuberculosis, Mycobacterium canettii, Mycobacterium africanum, Mycobacterium microti, M. bovis, Mycobacterium caprae and Mycobacterium pinnipedii) that are genetically very similar. From those species, M. tuberculosis is the most well known member, infecting more than one-third of the world's human population;
In the context of the present invention, tuberculosis is caused by various strains of mycobacteria, selected from the group comprising Mycobacterium tuberculosis, Mycobacterium canettii, Mycobacterium africanum, Mycobacterium microti, M. bovis, Mycobacterium caprae and Mycobacterium pinnipedii.

The present invention further provides inhibitors of mycobacterium virulence for use as a medicament.
It also relates to the use of a compound of general formula (I), (II), (IIA) or (III) in the preparation of a medicament. Preferably, the compound of general formula (I), (II), (IIA) or (III) is an inhibitor of mycobacterium virulence.

The present invention also provides inhibitors of mycobacterium virulence of general formula (I), for use in the treatment and/or prevention of tuberculosis.
Alternatively, the present invention provides inhibitors of mycobacterium virulence of general formula (IIA) or (II), for use in the treatment and/or prevention of tuberculosis.
The present invention also provides inhibitors of mycobacterial virulence of general formula (III), for use in the treatment and/or prevention of tuberculosis.
It also relates to the use of a compound of general formula (I), (II), (IIA) or (III) in the preparation of a medicament for treating and/or preventing tuberculosis. Preferably, the compound of general formula (I), (II), (IIA) or (III) is an inhibitor of mycobacterium virulence.

The present invention further provides a method of treating and/or preventing tuberculosis, said method comprises the administration of a therapeutically effective amount of an inhibitor of mycobacterium virulence of the present invention to a subject in need thereof.
As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder, such as tuberculosis. The term does not denote a particular age or sex. Thus, adult and newborn subjects, whether male or female, are intended to be covered.
The term “therapeutically effective amount” as used herein refers to an amount of at least one inhibitor of mycobacterium virulence or a pharmaceutical formulation thereof according to the invention that elicits the biological or medicinal response in animal or human that is being sought. The term includes the amount for the alleviation of the symptoms of the disease or condition being treated. The term also includes herein the amount of inhibitors of mycobacterium virulence sufficient to reduce and/or prevent the progression of the disease, namely tuberculosis, notably to reduce, inhibit and/or prevent the recurrence process of Tuberculosis.

The inhibitors of mycobacterium virulence, methods and uses according to the present invention are able to prevent, reduce or eradicate the dissemination of mycobacterium, selected from the group Mycobacterium tuberculosis, Mycobacterium canettii, Mycobacterium africanum, Mycobacterium microti, M. bovis, Mycobacterium caprae and Mycobacterium pinnipedii, in a subject. Preferably, the mycobacterium is Mycobacterium tuberculosis.

Interestingly, the Applicant demonstrates that the mycobacterium virulence inhibitors of the invention inhibit the secretion of the major mycobacterium virulence protein EsxA.
Thus, the present invention provides inhibitors of mycobacterium virulence that reduce ESX-1-dependent pathogenicity.

The present invention further provides a pharmaceutical composition comprising an inhibitor of mycobacterium virulence of formula (I), and a pharmaceutically acceptable carrier, diluent or excipient.
Alternatively, the present invention provides a pharmaceutical composition comprising an inhibitor of mycobacterium virulence of formula (IIA) or (II), and a pharmaceutically acceptable carrier, diluent or excipient.
The present invention also provides a pharmaceutical composition comprising an inhibitor of mycobacterial virulence of formula (III), and a pharmaceutically acceptable carrier, diluent or excipient.
As to the appropriate carriers, reference may be made to the standard literature describing these, e.g. to chapter 25.2 of Vol. 5 of “Comprehensive Medicinal Chemistry”, Pergamon Press 1990, and to “Lexikon der Hilfsstoffe für Pharmazie, Kosmetik und angrenzende Gebiete”, by H. P. Fiedler, Editio Cantor, 2002. The term “pharmaceutically acceptable carrier” means a carrier or excipient that is useful in preparing a pharmaceutical composition that is generally safe, and possesses acceptable toxicities. Acceptable carriers include those that are acceptable for veterinary use as well as human pharmaceutical use. A “pharmaceutically acceptable carrier” as used in the specification and claims includes both one and more than one such carrier.
The compounds of the invention, namely inhibitors of mycobacterium virulence of formula (I), (IIA), (II) or (III), that are used in the treatment and/or prevention of tuberculosis can be incorporated into a variety of formulations and medicaments for therapeutic administration.
More particularly, one or more compound(s) as provided herein can be formulated into pharmaceutical compositions by combination with appropriate, pharmaceutically acceptable carriers, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the compounds can be achieved in various ways, including oral, buccal, rectal, parenteral, intraperitoneal, intradermal, transdermal, intracranial and/or intratracheal administration. Moreover, the compound can be administered in a local rather than systemic manner, in a depot or sustained release formulation. The compounds can be formulated with common excipients, diluents or carriers, and compressed into tablets, or formulated as elixirs or solutions for convenient oral administration, or administered by the intramuscular or intravenous routes. The compounds can be administered transdermally, and can be formulated as sustained release dosage forms and the like. The compounds can be administered alone, in combination with each other, or they can be used in combination with other known compounds. Suitable formulations for use in the present invention are found in Remington's Pharmaceutical Sciences (Mack Publishing Company (1985) Philadelphia, Pa., 17th ed.), which is incorporated herein by reference. Moreover, for a brief review of methods for drug delivery, see, Langer, Science (1990) 249:1527-1533, which is incorporated herein by reference.
The amount of a compound as provided herein that can be combined with a carrier material to produce a single dosage form will vary depending upon the disease treated, the subject in need thereof, and the particular mode of administration. However, as a general guide, suitable unit doses for the compounds of the present invention can, for example, preferably contain between 0.1 mg to about 1000 mg, between 1 mg to about 500 mg, and between 1 mg to about 300 mg of the active compound. In another example, the unit dose is between 1 mg to about 100 mg. Such unit doses can be administered more than once a day, for example, 2, 3, 4, 5 or 6 times a day, but preferably 1 or 2 times per day, so that the total dosage for a 70 kg human adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area. A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release. It can be necessary to use dosages outside these ranges in some cases as will be apparent to those skilled in the art.
Optionally, the pharmaceutical composition of the present invention further comprises one or more additional active agents selected from the group of the mycobacterium virulence inhibitor of general formula I, IIA, II and/or III.

The present invention provides also a method wherein anti-virulence compounds and not growth inhibitory drugs are selected. A putative mycobacterium virulence inhibitor was defined as a hit compound that protected fibroblasts from Mtb-induced cell death in the Fibroblast survival assay (FSA) without affecting bacterial growth in the REMA. In particular, the influence of inhibitors of mycobacterium virulence on Mtb growth is verified against Mtb in the resazurin reduction microtiter assay (REMA).
It has been found that mycobacterium virulence inhibitors inhibit mycobacterial protein secretion of the ESX-1 secretion system.
Furthermore, mycobacterium virulence inhibitors of general formula I deregulate genes controlled by two-component regulatory systems.

Thus, the present invention provides a screening method for identifying inhibitors of mycobacterium virulence, said method comprising

a) Infecting eukaryotic cells and/or macrophages with wild-type Mtb-Erdman strain at high multiplicities of infection (MOI),

b) Contacting said infected eukaryotic cells and/or infected macrophages with an inhibitor to be screened,

c) Quantifying metabolic activity in said eukaryotic cells and/or macrophages,

wherein said inhibitor fulfills the following criteria:

i) protects said eukaryotic cells and/or macrophages from Mycobacterium tuberculosis (Mtb)-induced cell death during and after the exposure to the said inhibitor,

ii) does not influence Mtb growth, and

iii) either inhibits the histidine kinase MprB in Mtb or affects metal ion homeostasis in Mtb.
The present invention also provides a screening method for identifying inhibitors of mycobacterium virulence, said method comprising

a) Infecting MRC-5 lung fibroblasts and/or THP-1 macrophages with wild-type Mtb-Erdman strain at high multiplicities of infection (MOI),

b) Contacting said infected MRC-5 lung fibroblasts and/or infected THP-1 macrophages with an inhibitor to be screened,

c) Quantifying metabolic activity in said MRC-5 lung fibroblasts and/or THP-1 macrophages,

wherein said inhibitor fulfills the following criteria:

i) protects said MRC-5 lung fibroblasts and/or THP-1 macrophages from Mycobacterium tuberculosis (Mtb)-induced cell death during and after the exposure to the said inhibitor,

ii) does not influence Mtb growth, and

iii) either inhibits the histidine kinase MprB in Mtb or affects metal ion homeostasis in Mtb.

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.
In the case of conflict, the present specification, including definitions, will control. Unless defined otherwise, all technical and scientific terms used herein have the same meaning as is commonly understood by one of skill in art to which the subject matter herein belongs.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

The foregoing description will be more fully understood with reference to the following examples. Such examples, are, however, exemplary of methods of practicing the present invention and are not intended to limit the scope of the invention.

### EXAMPLES

### Example 1

### Experimental Procedures

### Culture Conditions of Bacterial Strains and Eukaryotic Cell Lines

Mycobacterial strains were routinely grown in Middlebrook 7H9 broth (supplemented with 0.2% glycerol, 10% ADC and 0.05% Tween-80) or in Sauton's medium for the analysis of culture filtrates. MRC-5 human lung fibroblasts were received from the Corriell Institute for Medical Research and grown in MEM-medium supplemented with 10% heat inactivated fetal bovine serum (FBS), 1% non-essential amino acids and 1 mM sodium pyruvate. THP-1 macrophages were grown in RPMI-medium supplemented with 10% FBS. Both cell lines were grown at 37° C. with 5% CO2.

### HTS

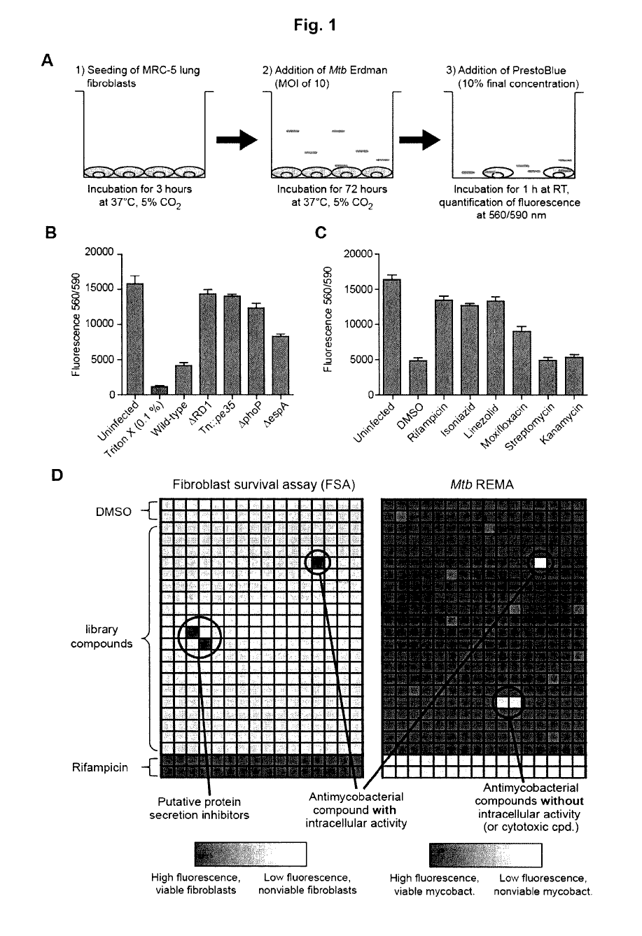

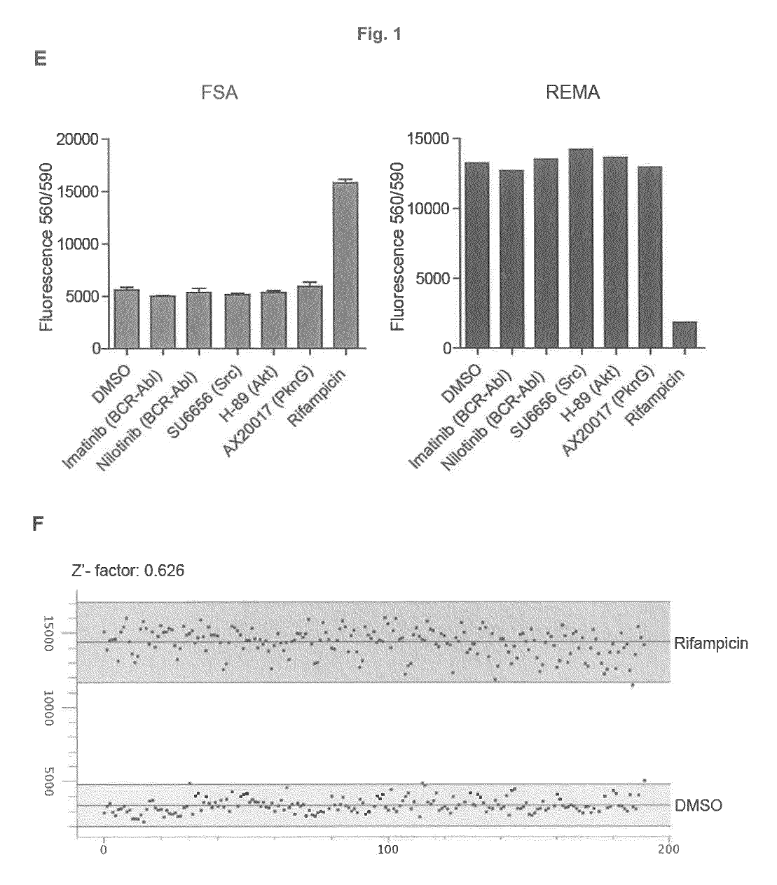

Library compounds were preplated into cellbind 384-well microplates (Corning) at a concentration of 50 μM in 5 μl of 5% DMSO. MRC-5 cells grown to late log phase were harvested and seeded at 4,000 cells/well in a volume of 35 μl into the plates using an automated microplate dispenser (multidrop combi, Thermo Scientific). Cells were allowed to adhere for 3 hours. Mid-logarithmic phase cultures of Mtb-Erdman were washed twice with complete 7H9 and added to the assay plates at an MOI of 10 in 10 μl of MEM medium. Plates were sealed and incubated at 37° C./5% CO2. Rifampicin was used as a control at 5 μg/ml, see FIG. 1 for assay plate layout. After 72 hours, the temperature of the plates was equilibrated to room temperature (RT) for 1 hour and 5 μl of Prestoblue cell viability reagent (Life Technologies) were added. After 1 hour at RT, fluorescence was measured in a Tecan infinite M200 plate reader (excitation 570 nm, emission 590 nm). By using this method, background fluorescence generated by the bacteria was negligible. REMA assays were performed in 7H9 broth using a starting OD of 0.0001, a 7 day incubation period and a final volume of 10% resazurin (0.025% w/v). Z′-factor determinations were performed as described (Zhang et al., 1999). Replicates were considered as hits if their values were superior to the mean of the negative control values plus 3 standard deviations. The final score was the mean value of the replicates.

### Immunoblots and Secretome Analysis

Protein preparation of mycobacteria and immunoblots were performed as described (Chen et al., 2013). In brief, 30 ml of bacteria grown to mid-logarithmic phase (OD600 of 0.6 to 0.7) in Sauton's medium supplemented with 0.05% Tween 80 were centrifuged and resuspended in Sauton's medium without Tween. Compounds were added at concentrations as indicated and cells were grown further at 37° C. with shaking for 4 days. Cultures were harvested by centrifugation to obtain culture filtrates and cell pellets. Culture filtrates were concentrated 100-fold in 5-kDa cutoff Vivaspin columns (Sartorius). Cell lysates were prepared by bead beating bacterial pellets in lysis buffer with 100-μm glass beads.

For immunoblot analysis, 5 μg of protein were resolved by SDS-gel electrophoresis and transferred to nitrocellulose membranes. Membranes were blocked with TBS-buffer (3% milk powder) and incubated overnight with the desired primary antibody diluted in TNT-buffer supplemented with 1% BSA fraction V. Membranes were washed with TNT, incubated with the appropriate secondary antibody in TNT-BSA, washed again with TNT, and developed. GroEL2 was used as a lysis control for culture filtrates and as a loading control for cell lysates.

For the secretome analysis 10 μg of protein was reconstituted in 200 μl of 4 M Urea, 10% acetonitrile and buffered with Tris-HCl pH 8.5 to a final concentration of 30 mM. Proteins were reduced in 10 mM dithioerythritol (DTE) at 37° C. for 60 min. and then alkylated in 40 mM iodoacetamide at 37° C. for 45 min. Reactions were quenched by addition of DTE to a final concentration of 10 mM. First, protein digestion was performed using Lys-C (1:50 enzyme:protein) for 2 hours at 37° C. The lysates were then diluted 5-fold and a second digestion was performed overnight at 37° C. using mass spectrometry grade trypsin gold (1:50 enzyme:protein) and 10 mM CaCl2. Reactions were stopped by addition of 8 μl of pure formic acid and peptides were concentrated by vacuum centrifugation to a final volume of 70 μl.

RNA Extraction, qRT-PCR and RNA-seq

For the transcriptomic studies, bacteria were grown under the same conditions as for protein secretion assays. Drug exposure time was 8 hours with 5 μM of compound for RNA-seq experiments and confirmatory qRT-PCR. RNA was extracted with Trizol (Invitrogen) and treated with DNase I (Roche) prior to library preparation or generation of the cDNA template. cDNA was synthesized using the RevertAid First Strand cDNA Synthesis Kit (Fermentas) using random hexamer primer. cDNA corresponding to 10 ng of input RNA was used in each RT-PCR reaction supplemented with specific primer pairs (200 nM each) listed in Table S4 and SYBR-Green master mix (Applied Biosystems). Quantitative RT-PCR reactions were performed with the 7900HT Fast Real-Time PCR System (Applied Biosystems) with the following parameters: 50° C. for 2 min, 95° C. for 10 min, followed by 40 cycles of 95° C. for 15 s and 60° C. for 60 s. Melt curve analysis was used to confirm specific amplification for each primer pair. Unpaired Student's T-tests were used for statistical analyses.

For the RNA-seq library preparation 100 ng of total RNA were used in the TruSeq Stranded mRNA LT kit, according to the instructions provided by the manufacturer (Illumina). A small aliquot was analyzed on Qubit and Fragment Analyzer prior to sequencing on Illumina HiSeq using the TruSeq SR Cluster Generation Kit v3 and TruSeq SBS Kit v3. Data were processed with the Illumina Pipeline Software v1.82. RNA-seq data were deposited in the Gene Expression Omnibus (GEO) server at the National Center for Biotechnology Information (NCBI).

### Quantification of Intracellular ATP-Levels and EtBr Uptake Assays.

Bacteria were grown for 24 hours in the presence of test compounds. The BacTiter-Glo microbial cell viability reagent (Promega) was used for the quantification of ATP according to the recommendations of the manufacturer. For EtBr uptake assays, bacteria were washed with PBS containing 0.05% Tween 80, OD600 was adjusted to 0.4 and 100 μl were pipetted into black 96 well plates. EtBr was added (4 μM final concentration) and fluorescence was read every 2 min. at 545/600 nm. Unpaired Student's T-tests were used for statistical analyses.

### Fluorescence Microscopy

THP-1 macrophages were activated on round 9 mm cover slips in 24 well plates (105 cells/well) with 100 nM of phorbol-12-myristate-13-acetate for 72 hours. For the quantification of intracellular Mtb Erdman-GFP, macrophages were infected at an MOI of 2 for 12 hours. Cells were washed several times to remove unphagocytosed bacteria and fresh medium containing compounds or DMSO was added. After incubation for four days, the cells were washed and fixed with 4% paraformaldehyde/PBS and stained with Dapi-Fluoromount-G (SouthernBiotech). Images were acquired on a Zeiss LSM 700 using ZEN imaging software and Fiji processing software. At least forty fields of three separate monolayers were collected for image processing and statistical analysis. For the intracellular localization studies cells were prepared as described above and infected at an MOI of 0.5. After 12 hours extracellular bacteria were removed by washing with PBS and fresh medium containing compounds or DMSO was added. Incubation continued for a total of 7 days with replacement of media plus compounds after 3 days. Fresh media containing 50 nM of Lysotracker Red (Life Technologies) was added for 2 hours. Cells were washed and fixed as described above. Colocalization rates of GFP-fluorescing phagosomes and Lysotracker Red were determined by analyzing >100 phagosomes from at least three separate monolayers. Unpaired Student's T-tests were used for statistical analyses.

### Protein Purification and Kinase Inhibitor Assay

Protein purification was performed as described recently (Rybniker et al., 2014). For autophosphorylation assays, MprB lacking its N-terminal transmembrane domain was incubated with [γ-32P]ATP (10 mCi/ml, 3,000 Ci/mmol) in 50 mM Tris-HCl (pH 7.5), 50 mM KCl and 20 mM MnCl2 for 1 hour. Reactions were stopped by adding SDS-loading dye and heating the samples for 5 min at 80° C. followed by separation using SDS-PAGE. Gels were either stained with Coomassie brilliant blue or dried for 2 hours at 60° C. in a model 583 gel dryer (Biorad) followed by exposure to X-ray film overnight or counting of 32P-incorporation into band equivalents using a LS6500 scintillation counter (Beckman-Coulter). For kinase inhibitor assays, compounds were pre-incubated with MprB for 3 hours prior to addition of [γ-32P]ATP. Unpaired Student's T-tests were used for statistical analyses.

### Culture Conditions and REMA Assay of Non-Mtb Strains.

Mycobacterium strains (Mycobacterium bovis BCG, M. marinum strain M, M. smegmatis MC2155) were grown in 7H9 broth (Difco) supplemented with Middlebrook albumin-dextrose-catalase (ADC) enrichment, 0.2% glycerol, 0.05% Tween 80. Bacillus subtilis, Candida albicans, Corynebacterium glutamicum ATCC13032, Micrococcus luteus, Pseudomonas putida, Salmonella typhimurium and Staphylococcus aureus were grown in Luria broth base (Sigma). Corynebacterium diphtheriae, Enterococcus faecalis, Listeria monocytogenes and Pseudomonas aeruginosa were grown in brain heart infusion broth (Difco). Two-fold serial dilutions of each test compound were prepared in 96-well plates containing bacteria in a total volume of 100 μl and then incubated at 37° C. or 30° C. (depend on the strain) before addition of 10 μl of 0.025% resazurin. After incubation, fluorescence of the resazurin metabolite resorufin was determined (excitation at 560 nm and emission at 590 nm, Gain 80) by using a TECAN Infinite M200 microplate reader.

### Dimethyl Labeling and SAX Fractionation of Digested Culture Filtrate Proteins

After digestions, samples were dimethyl-labeled as described previously (Boersema et al., 2009). In brief, culture filtrates from bacteria treated with DMSO were labeled with light dimethyl reactants (CH2O+NaBH3CN) and Culture filtrates from bacteria treated with BBH7 were labeled with medium reactants (CD2O+NaBH3CN). In the “reverse” experiment, the labelling of the culture filtrates from bacteria treated with DMSO and BBH7 samples were reversed. As a final step of labeling the procedure, samples were mixed in a 1:1 (Light:Medium) and lyophilized.
SAX fractionation was performed as previously described with minor modifications (Wisniewski et al., 2009). Stage Tips were prepared by placing six layers of a 3M Empore™ anion exchange disk (3M) into a P200 pipette tips. SAX buffers were freshly prepared and titrated (pH 2, 4, 5, 6, 8, 11) with NaOH. Tips were first conditioned successively with 100% Methanol, 1M NaOH and Phosphoric acid buffer (pH 11). Samples were reconstituted in SAX buffer (pH 11) and loaded into the conditioned tips. The loading flow-through as well as the pH step elutions (in decreasing order of pH) were on-line captured on Empore™ C18 stage tips. Each collected fraction was washed with 0.1% TFA and eluted with acidified high organic content solvent. Eluted fractions were finally dried by vacuum centrifugation and used for LC-MS/MS analysis.

### Mass Spectrometry and Data Analysis:

Each SAX fraction was resuspended in 2% acetonitrile, 0.1% FA and loaded on a capillary pre-column (Magic AQ C18; 3 μm by 200 Å; 2 cm×100 μm ID). Separations were performed on a C18 tip-capillary column (Nikkyo Technos Co; Magic AQ C18; 3 μm by 100 Å; 15 cm×75 μm) using a Dionex Ultimate 3000 RSLC nano UPLC system. Data were acquired in data-dependent mode (over a 4 hr acetonitrile 2-42% gradient) on an Orbitrap Elite Mass spectrometer. Acquired RAW files were processed using MaxQuant version 1.3.0.5 (Cox et al., 2009) and its internal search engine Andromeda (Cox et al., 2011). The Mtb strain H37Rv R26 database (http://tuberculist.epfl.ch/) (Lew et al., 2011) was used for the search and MaxQuant default identification settings were applied in combination with specific dimethyl labeling parameters. Search results were filtered with a false-discovery rate of 0.01. Known contaminants and reverse hits were removed before statistical analysis. Relative quantification within different conditions was obtained calculating the significance B values for each of the identified proteins using Perseus (Cox et al., 2009).

### Genome Annotation and RNA-seq Data Analysis

All analyses in this study were carried out using the M. tuberculosis H37Rv annotation from the TubercuList database (http://tuberculist.epfl.ch/) (Lew et al., 2011). There are 4019 protein coding sequences (CDS) currently annotated in the genome, 73 genes encoding for stable RNAs, small RNAs and tRNAs. In order to quantify protein occupancy and transcription across the entire genome, 3080 intergenic regions (regions flanked by two non-overlapping CDS) were included, resulting in a total of 7172 features.
The single-ended sequence reads generated from RNA-seq experiments were aligned to the M. tuberculosis H37Rv genome (NCBI accession NC_000962.2) using Bowtie2 with default parameters (Langmead and Salzberg, 2012). Read counts for all annotated features were obtained with the htseq-count program (http://www.huber.embl.de/users/anders/HTSeq/doc/count.html). Regions where genes overlapped were excluded from counting. Reads spanning more than one feature were counted for each feature. Since the RNA library was strand-specific, the orientation of sequence reads had to correspond to the orientation of annotated features to be counted. Analysis of differential gene expression was carried out using the DESeq package (Anders and Huber, 2010).

### Cloning and Purification of His6-Tagged MprB

Cloning of the mprB PCR-product into pQE80L (Qiagen) was performed using the In-Fusion PCR Cloning kit (Clontech). Two litres of mid-log phase E. coli BL21 (DE3) culture were induced with 0.5 mM isopropyl β-d-thiogalactoside (IPTG) and incubated for 12 hours at 16° C. cells were lysed in lysis buffer (50 mM Tris pH 8, 500 mM NaCl, 5 mM imidazole, 10% glycerol, 1% Tween 20) using a French press. After clearance by centrifugation, the lysates were incubated with 1 g of PrepEase resin (USB, Cleveland, USA) for 1 hour at 4° C. followed by separation on a PolyPrep chromatography column (Biorad). The resin was washed with two column volumes of buffer containing 10 mM imidazole and eluted with 250 mM imidazole. After dialysis against 25 mM Tris pH 7.5 and 200 mM NaCl the protein was further purified by gel filtration on a HiLoad 16/60 Superdex 200 column (Amersham Biosciences).

### Data Processing for Intracellular Quantification of Bacteria Using Confocal Microscopy

For quantification of intracellular bacteria the DAPI-channel was filtered using a median filter of 2 pixels (radius), and a Gaussian blur with a sigma of 2 pixels. Afterwards, an automatic threshold using Huang's fuzzy thresholding method (Fiji, “Huang” auto threshold) was applied on this modified image of the DAPI-channel and an automatic threshold using Tsai's thresholding method (Fiji, “Moments” auto threshold) was applied on the bacteria-channel. Finally, the area of each segmented image was measured. Areas or their ratio can be plotted and are indicative of the bacterial load within macrophage.

### Synthesis of BTP15 (5 -Bromo-2-(cyclopropanecarbonyl-amino)-6-hydroxy-benzo[b]thiophene-3-carboxylic acid amide)

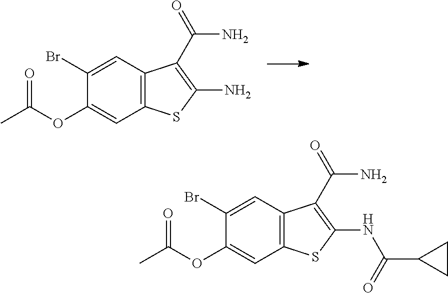 [SMILES: CC(=O)Oc1cc2sc(N)c(C(N)=O)c2cc1Br ; CC(=O)Oc1cc2sc(NC(=O)C3CC3)c(C(N)=O)c2cc1Br] [0234] The solution of 0.33 g (1 mmol) acetic acid 2-amino-5-bromo-3-carbamoyl-benzo[b]thiophen-6-yl ester (HU P1300338) in 10 cm3 pyridine at 0° C. was treated drop wise with 0.12 g, 0.10 cm3 (1.10 mmol) cylopropylcarbonylchloride. The reaction mixture was stirred at room temperature (RT) for five hours, and then was evaporated under vacuum. The residue was stirred in 15 cm3 1 N water solution of hydrochloride acid at RT for 30 minutes, then the product was filtered off and was washed with water. The crude product was refluxed in 10 cm3 ethanol for half an hour, it was cooled to 0° C. and the pure product was filtered off.
Yield: 0.30 g (75%)
1H-NMR (DMSO-d6): 11.80 (s, 1H), 7.95 (s, 1H), 7.70 (bs, 2H), 7.35 (s, 1H), 1.98 (m, 1H), 0.91 (m, 4H) ppm.
LC-MS: M−=395

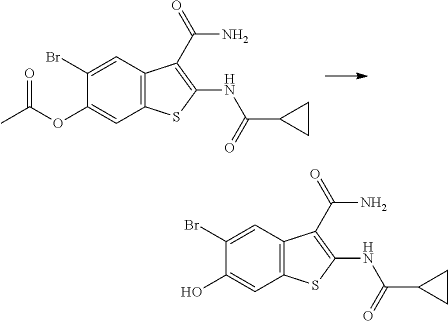

The solution of 0.20 g (0.50 mmol) acetic acid 5-bromo-3-carbamoyl-2-(cyclopropanecarbonyl-amino)-benzo[b]thiophen-6-yl ester in 30 cm3 methanol at RT was treated in one portion with a 1.00 cm3 (2.00 mmol) water solution of sodium hydroxide. The reaction mixture was stirred at RT for two hours, and then was evaporated under vacuum. The residue was stirred in 15 cm3 1 N water solution of hydrochloride acid at RT for half an hour, then the product was filtered off and was washed with water. The crude product was refluxed in 10 cm3 acetonitrile for half an hour, it was cooled to 0° C. and the pure product was filtered off.
Yield: 0.14 g (77%)
1H-NMR (DMSO-d6): 11.80 (s, 1H), 10.32 (s, 1H), 7.98 (s, 1H), 7.74 (bs, 2H), 7.38 (s, 1H), 1.98 (m, 1H), 0.91 (m, 4H) ppm.
LC-MS: M−=353

### Synthesis of BBH7 (4)

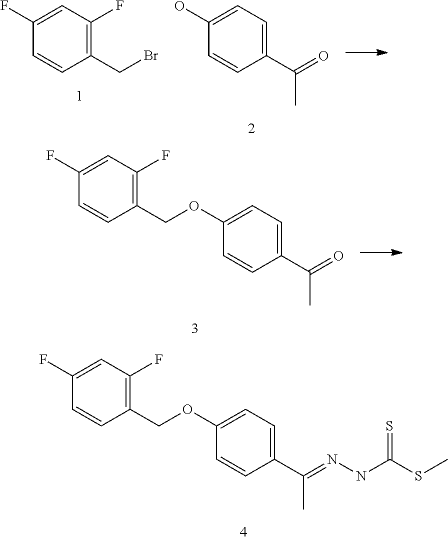

### 1-[4-(2,4-Difluoro-benzyloxy)-phenyl]-ethanone (3)

The mixture of 2,4-difluorobenzyl bromide (1, 6.00 g, 29 mmol), acetone (45 ml), potassium carbonate (2.18 g, 16 mmol), potassium iodide (100 mg) and 4′-hydroxyacetophenone (2, 4.08 g, 30 mmol) was stirred at reflux temperature for 24 hours. The inorganic salts were filtered off, washed with acetone then the filtrate was evaporated in vacuum. The residue was taken up in the mixture of chloroform (30 ml) and aqueous sodium hydroxide solution (10 wt %, 20 ml). The two layers were separated; the aqueous layer was extracted two times with chloroform (2×20 ml). The organic layers were combined, washed with water, dried on sodium sulfate, and evaporated in vacuum. The residue was solidified under hexane. The precipitate was filtered washed with hexane then dried on air. Thus 6.64 g of the title compound (3) was obtained. Yield: 87%
C15H12F2O2, Mw=262.26, Exact Mass=262.08
LC-MS purity: 99% m/z 263 [M]+, Rt. 4.24 min.
1H-NMR in DMSO-d6 δ: 7.94 (dm, J=8.8 Hz, 2H), 7.65 (ddd, J=8.7, 8.7 and 6.8 Hz, 1H), 7.32 (ddd, J=10.6, 9.4 and 2.5 Hz, 1H), 7.15 (dddd, J=8.7, 8.7, 2.5 and 1.0 Hz, 1H), 7.14 (dm, J=8.8 Hz, 2H), 5.21 (s, 2H), 2.52 (s, 3H)

### N′-{1-[4-(2,4-Difluoro-benzyloxy)-phenyl]ethylidene}-hydrazinecarbodithioic acid methyl ester (4)

The mixture of 1-[4-(2,4-difluoro-benzyloxy)-phenyl]-ethanone (3, 1.57 g, 6.00 mmol), hydrazinecarbodithioic acid methyl ester (732 mg, 6.00 mmol) and acetic acid (20 ml) was stirred at room temperature for 24 hours. The precipitate was filtered off, washed with acetic acid then with diisopropyl ether and dried under vacuum. Thus 1.68 g of the title compound (4) was obtained. Yield: 76%
C17H16F2N2OS2, Mw=366.45, Exact Mass=366.07
LC-MS purity: 99%, m/z 365 [M−H]−, 367 [M]+ Rt. 4.90 min.
1H-NMR in DMSO-d6 δ: 12.35 (s, 1H), 7.83 (dm, J=8.8 Hz, 2H), 7.64 (ddd, J=9.0, 8.3 and 6.4 Hz, 1H), 7.31 (ddd, J=10.1, 10.0 and 1.9 Hz, 1H), 7.14 (dddd, J=9.0, 8.3, 1.9 and 1.0 Hz, 1H), 7.09 (dm, J=8.8 Hz, 2H), 5.17 (s, 2H), 2.50 (s, 3H), 2.35 (s, 3H)
13C-NMR δ: 199.5, 162.0 (JC,F=247.0 and 12.8 Hz), 160.5 (JC,F=248.0 and 12.8 Hz),

159.8, 151.7, 132.4 (JC,F=10.3 and 5.2 Hz), 130.2, 128.3, 120.2 (JC,F=14.9 and 3.8 Hz), 114.8, 111.8 (JC,F=21.3 and 3.6 Hz), 104.2 (JC,F=25.6 and 25.6 Hz), 63.4, (JC,F=2.3 Hz), 17.1, 14.6
The signal assignation is based on HSQC and HMBC experiments.
The E isomer is proven by crosspeaks between the NH (12.35 ppm) and the C—CH3 (2.35 ppm) signals observed in the ROESY spectrum.

### Synthesis of Mycobacterium Virulence Inhibitors of General Formula IIA

### Schematic Synthesis Pathway:

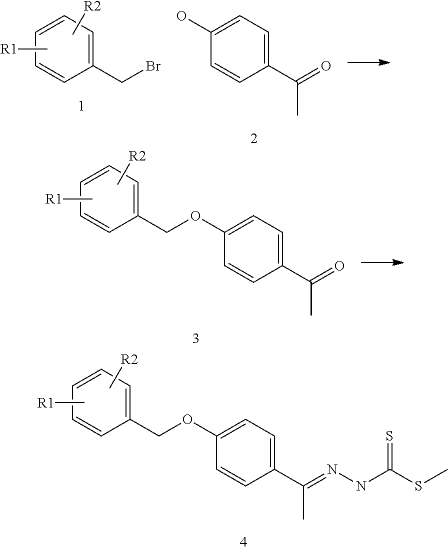

| Code | Structure | LCMS | NMR |
|---|---|---|---|
| 14467 | 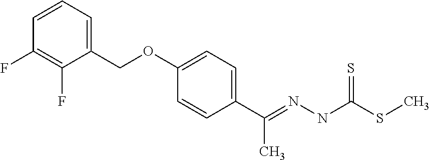 [SMILES: CSC(=S)N/N=C(\C)c1ccc(OCc2cccc(F)c2F)cc1] | C17H16F2N2OS2 Mw = 366.45 LCMS purity: 95% m/z 367 [M − H]⁺, Rt. 3.56 min | 12.39 (s, 1H); 7.84 (dm, J = 8.8 Hz, 2H); 7.46 (dddd, J = 10.0, 8.5, 8.0 & 1.4 Hz, 1H); 7.39 (dddd, J~6.5, 6.5, 1.4 & 1.4 Hz, 1H); 7.26 (dddd, J = 8.0, 8.0, 5.1 & 1.4 Hz, 1H); 7.44 (dm, J = 8.8 Hz, 2H); 5.26 (s, 2H); 2.50 (s, 3H); 2.35 (s, 3H) |
| 14468 | 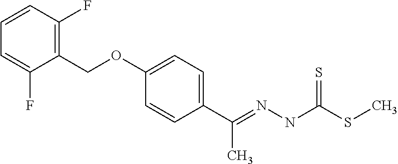 [SMILES: CSC(=S)N/N=C(\C)c1ccc(OCc2c(F)cccc2F)cc1] | C17H16F2N2OS2 Mw = 366.45 LCMS purity: 95% m/z 365 [M − H]⁻, 367 [M − H]⁺, Rt. 3.53 min | 12.40 (s, 1H); 7.84 (dm, J = 8.8 Hz, 2H); 7.54 (tt, J = 8.5 & 6.6 Hz, 1H); 7.20 (ddm, J = 8.5 & 7.6 Hz, 2H); 7.11 (dm, J = 8.8 Hz, 2H); 5.18 (s, 2H); 2.50 (s, 3H); 2.36 (s, 3H) |
| 14469 | 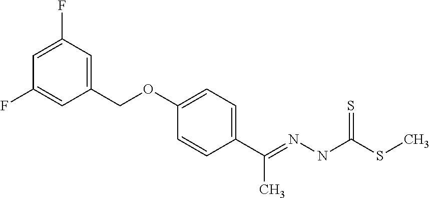 [SMILES: CSC(=S)N/N=C(\C)c1ccc(OCc2cc(F)cc(F)c2)cc1] | C17H16F2N2OS2 Mw = 366.45 LCMS purity: 99% m/z 365 [M − H]⁻, 367 [M − H]⁺, Rt. 3.58 min | 12.39 (s, 1H); 7.84 (dm, J = 8.8 Hz, 2H); 7.15- 7.25 (ovl. m, 3H); 7.08 (dm, J = 8.8 Hz, 2H); 5.20 (s, 2H); 2.50 (s, 3H); 2.35 (s, 3H) |
| 14472 | 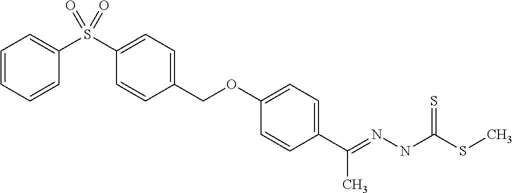 [SMILES: CSC(=S)N/N=C(\C)c1ccc(OCc2ccc(S(=O)(=O)c3ccccc3)cc2)cc1] | C23H22N2O3S3 Mw = 470.64 LCMS purity: 94% m/z 469 [M − H]⁻, 471 [M − H]⁺, Rt. 3.53 min | 12.38 (s, 1H); 7.99 (dm, J~8.0 Hz, 2H); 7.96 (dm, J~8.0 Hz, 2H); 7.81 (dm, J = 8.8 Hz, 2H); 7.69 (tm, J~7.5 Hz, 1H); 7.68 (dm, J~8.0 Hz, 2H); 7.63 (ddm, J~8.0 & 7.5 Hz, 2H); 7.05 (dm, J = 8.8 Hz, 2H); 5.26 (s, 2H); 2.49 (s, 3H); 2.33 (s, 3H) |
| 14475 | 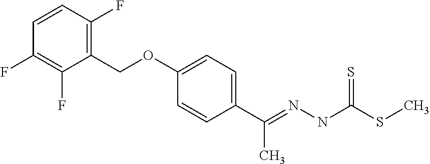 [SMILES: CSC(=S)N/N=C(\C)c1ccc(OCc2c(F)ccc(F)c2F)cc1] | C17H15F3N2OS2 Mw = 384.45 LCMS purity: 97% m/z 383 [M − H]⁻, 385 [M − H]⁺, Rt. 3.55 min | 12.41 (s, 1H); 7.85 (dm, J = 8.8 Hz, 2H); 7.62 (dddd, J = 9.5, 9.5, 9.5 & 5.1 Hz, 1H); 7.25 (dddd, J = 9.5, 8.5, 3.8 & 2.2 Hz, 1H); 7.11 (dm, J = 8.8 Hz, 2H); 5.22 (s, 2H); 2.50 (s, 3H); 2.36 (s, 3H) |
| 14476 | 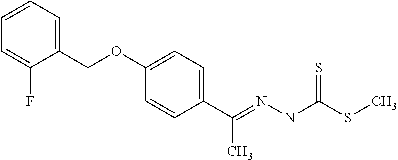 [SMILES: CSC(=S)N/N=C(\C)c1ccc(OCc2ccccc2F)cc1] | C17H17FN2OS2 Mw = 348.46 LCMS purity: 92% m/z 347 [M − H]⁻, 349 [M − H]⁺, Rt. 3.54 min | 12.38 (s, 1H); 7.84 (dm, J = 8.8 Hz, 2H); 7.58 (ddd, J~8.0, 8.0 & 1.5 Hz, 1H); 7.44 (dddd, J~7.5, 7.0, 6.5 & 1.5 Hz, 1H); 7.23-7.31 (ovl. m, 2H); 7.10 (dm, J = 8.8 Hz, 2H); 5.20 (s, 2H); 2.50 (s, 3H); 2.35 (s, 3H) |
| 14478 | 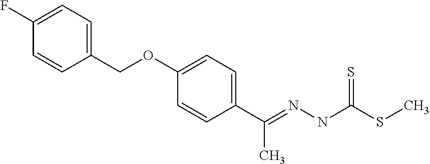 [SMILES: CSC(=S)N/N=C(\C)c1ccc(OCc2ccc(F)cc2)cc1] | C17H17FN2OS2 Mw = 348.46 LCMS purity: 99% m/z 347 [M − H]⁻, 349 [M − H]⁺, Rt. 3.53 min | 12.38 (s, 1H); 7.82 (dm, J = 8.8 Hz, 2H); 7.45 (ddm, J = 8.8 & 5.7 Hz, 2H); 7.17 (ddm, J = 8.8 & 8.8 Hz, 2H); 7.07 (dm, J = 8.8 Hz, 2H); 5.15 (s, 2H); 2.50 (s, 3H); 2.35 (s, 3H) |
| 14481 | 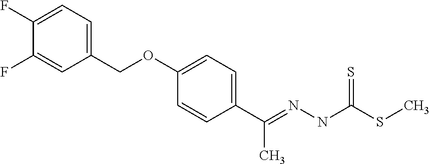 [SMILES: CSC(=S)N/N=C(\C)c1ccc(OCc2ccc(F)c(F)c2)cc1] | C17H16F2N2OS2 Mw = 366.45 LCMS purity: 97% m/z 365 [M − H]⁻, 367 [M − H]⁺, Rt. 3.57 min | 12.38 (s, 1H); 7.82 (dm, J = 8.8 Hz, 2H); 7.55 (ddd, J = 11.5, 8.0 & 2.1 Hz, 1H); 7.46 (ddd, J = 10.7, 8.7 & 8.4 Hz, 1H); 7.33 (dddd, J = 8.4, 4.5, 2.1 & 1.2 Hz, 1H); 7.07 (dm, J = 8.8 Hz, 2H); 5.15 (s, 2H); 2.50 (s, 3H); 2.34 (s, 3H) |
| 14482 | 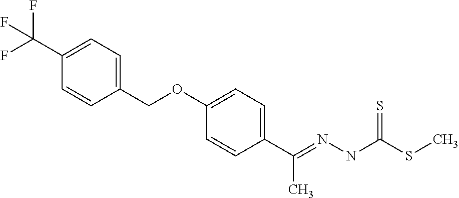 [SMILES: CSC(=S)N/N=C(\C)c1ccc(OCc2ccc(C(F)(F)F)cc2)cc1] | C18H17F3N2OS2 Mw = 398.47 LCMS purity: 99% m/z 397 [M − H]⁻, 399 [M − H]⁺, Rt. 3.63 min | 12.38 (s, 1H); 7.83 (dm, J = 8.8 Hz, 2H); 7.77 (dm, J = 8.2 Hz, 2H); 7.68 (dm, J = 8.2 Hz, 2H); 7.08 (dm, J = 8.8 Hz, 2H); 5.29 (s, 2H); 2.50 (s, 3H); 2.34 (s, 3H) |
| 14483 | 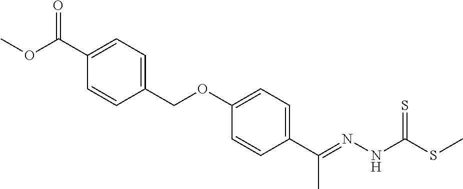 | C19H20N2O3S2 Mw = 388.51 LCMS purity: 95% m/z 387 [M − H]⁻, 389 [M − H]⁺, Rt. 3.54 min | 12.38 (s, 1H); 7.99 (dm, J = 8.2 Hz, 2H); 7.82 (dm, J = 8.8 Hz, 2H); 7.60 (dm, J = 8.2 Hz, 2H); 7.08 (dm, J = 8.8 Hz, 2H); 5.27 (s, 2H); 3.86 (s, 3H); 2.50 (s, 3H); 2.34 (s, 3H) |

### Preparation of Thiosemicarbazon Derivatives of General Formula (IIA)

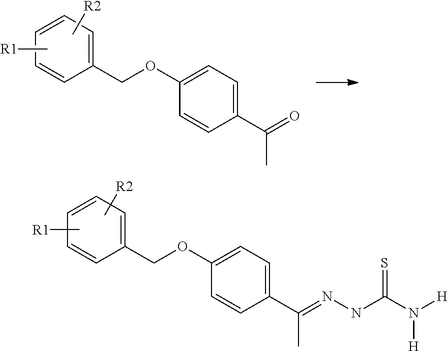 [0257] 131 mg (0.5 mmol) 1-[4-(2,4-Difluoro-benzyloxy)-acetophenon and 46 mg (0.5 mmol) thiosemicarbazide was refluxed in 1.5 ml ethyl alcohol for 2 days. The mixture was evaporated, then the residue was solidified under diisopropyl ether. The precipitate was filtered, washed with 50% aeous ethyl alcohol, methanol and diisopropyl ether. Thus, 90 mg appropriate thiosemicarbazon derivative was obtained (Yield: 54%).
If necessary, the raw product could be purified by chromatography on TLC plate (eluents: hexane-ethyl acetate=7:3). The pure product was solidified under diisopropyl ether.

| Code | Structure | LCMS | NMR |
|---|---|---|---|
| 29985 | 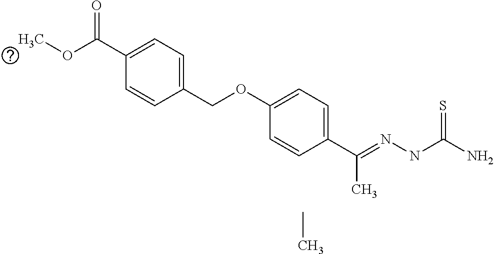 | C16H15F2N3OS Mw = 335.38 LCMS purity: 98% m/z 334 [M − H]⁻, 336 [M − H]⁺, Rt. 4.01 min Yield: 54% | 10.10 (s, 1H); 8.18 (br. s, 1H); 7.89 (dm, J = 8.8 Hz, 2H); 7.88 (br. s, 1H); 7.63 (ddd, J = 8.6, 8.6 & 6.7 Hz, 1H); 7.30 (ddd, J = 10.4, 9.5 & 2.6 Hz, 1H); 7.13 (dddd, J = 8.6, 8.6, 2.6 & 0.8 Hz, 1H); 7.02 (dm, J = 8.8 Hz, 2H); 5.15 (s, 2H); 2.26 (s, 3H) |
| 30650 | 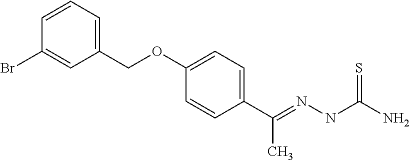 [SMILES: C/C(=N\NC(N)=S)c1ccc(OCc2cccc(Br)c2)cc1] | C16H16BrN3OS Mw = 378.29 LCMS purity: 95% m/z 376 [M − H]⁻, 378 [M − H]⁺, Rt. 4.31 min | 10.09 (s, 1H); 8.18 (br. s, 1H); 7.89 (dm, J = 8.8 Hz, 2H); 7.87 (br. s, 1H); 7.67 (dd, J = 1.5 & 1.5 Hz, 1H); 7.53 (dm, J = 7.7 Hz, 1H); 7.46 (dm, J = 7.8 Hz, 1H); 7.36 (dd, J = 7.8 & 7.7 Hz, 1H); 7.00 (dm, J = 8.8 Hz, 2H); 5.17 (s, 2H); 2.25 (s, 3H) |
| 30651 | 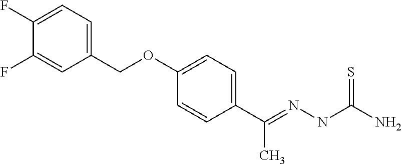 [SMILES: C/C(=N\NC(N)=S)c1ccc(OCc2ccc(F)c(F)c2)cc1] | C16H15F2N3OS Mw = 335.38 LCMS purity: 94% m/z 334 [M − H]⁻, 336 [M − H]⁺, Rt. 4.07 min | 10.10 (s, 1H); 8.19 (br. s, 1H); 7.89 (dm, J = 8.8 Hz, 2H); 7.87 (br. s, 1H); 7.54 (ddd, J = 11.5, 8.0 & 2.0 Hz, 1H); 7.46 (ddd, J = 10.7, 8.7 & 8.4 Hz, 1H); 7.32 (dddd, J = 8.4, 4.5, 2.0 & 1.2 Hz, 1H); 7.00 (dm, J = 8.8 Hz, 2H); 5.14 (s, 2H); 2.26 (s, 3H) |
| 30652 | 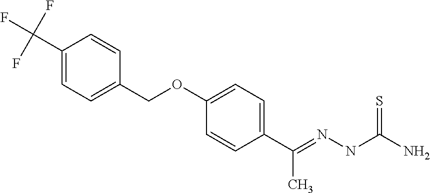 [SMILES: C/C(=N\NC(N)=S)c1ccc(OCc2ccc(C(F)(F)F)cc2)cc1] | C17H16F3N3OS Mw = 367.40 LCMS purity: 95% m/z 366 [M − H]⁻, 368 [M − H]⁺, Rt. 4.35 min | 10.10 (s, 1H); 8.19 (br. s, 1H); 7.89 (dm, J = 8.8 Hz, 2H); 7.87 (br. s, 1H); 7.77 (dm, J = 8.2 Hz, 2H); 7.67 (dm, J = 8.2 Hz, 2H); 7.01 (dm, J = 8.8 Hz, 2H); 5.28 (s, 2H); 2.26 (s, 3H) |
| 30653 | 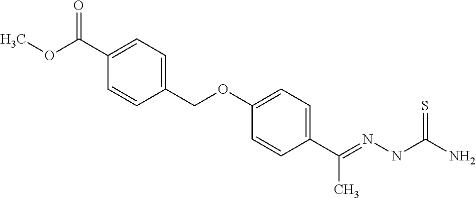 [SMILES: COC(=O)c1ccc(COc2ccc(/C(C)=N/NC(N)=S)cc2)cc1] | C18H19N3O3S Mw = 357.43 LCMS purity: 99% m/z 356 [M − H]⁻, 358 [M − H]⁺, Rt. 3.83 min | 10.07 (s, 1H); 8.16 (br. s, 1H); 7.98 (dm, J = 8.2 Hz, 2H); 7.88 (dm, J = 8.8 Hz, 2H); 7.86 (br. s, 1H); 7.60 (dm, J = 8.2 Hz, 2H); 7.01 (dm, J = 8.8 Hz, 2H); 5.26 (s, 2H); 3.86 (s, 3H); 2.26 (s, 3H) |
| 30654 | 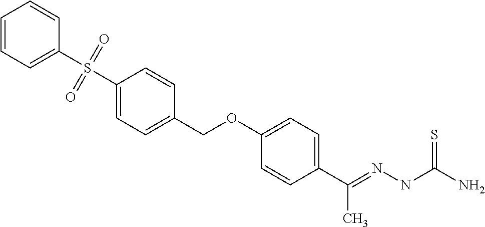 [SMILES: C/C(=N\NC(N)=S)c1ccc(OCc2ccc(S(=O)(=O)c3ccccc3)cc2)cc1] | C22H21N3O3S2 Mw = 439.56 LCMS purity: 95% m/z 438 [M − H]⁻, 440 [M − H]⁺, Rt. 3.91 min | 10.09 (s, 1H); 8.17 (br. s, 1H); 7.98 (dm, J = 7.5 Hz, 2H); 7.96 (dm, J = 7.0 Hz, 2H); 7.87 (dm, J = 8.8 Hz, 2H); 7.85 (br. s, 1H); 7.69 (tm, J = 7.5 Hz, 1H); 7.68 (dm, J = 7.5 Hz, 2H); 7.62 (ddm, J = 7.5 & 7.0 Hz, 2H); 7.01 (dm, J = 8.8 Hz, 2H); 5.25 (s, 2H); 2.24 (s, 3H) |
| 30655 | 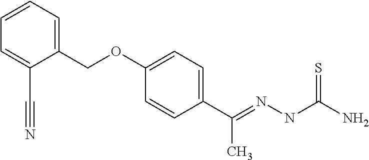 [SMILES: C/C(=N\NC(N)=S)c1ccc(OCc2ccccc2C#N)cc1] | C17H16N4OS Mw = 324.41 LCMS purity: 94% m/z 323 [M − H]⁻, 325 [M − H]⁺, Rt. 3.66 min | 10.11 (s, 1H); 8.19 (br. s, 1H); 7.91 (dm, J = 8.8 Hz, 2H); 7.90 (m, 1H); 7.88 (br. s, 1H); 7.71-7.80 (ovl. m, 2H); 7.58 (m, 1H); 7.04 (dm, J = 8.8 Hz, 2H); 5.30 (s, 2H); 2.27 (s, 3H) |

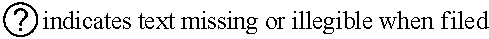

### Preparation of N-allyl-thiosemicarbazon Derivatives of General Formula (IIA)

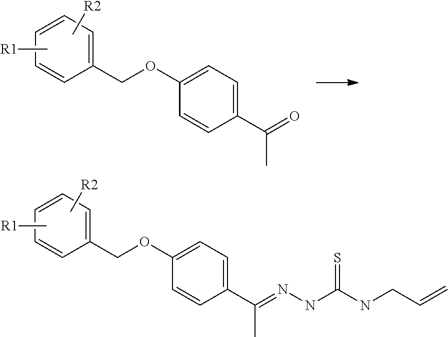 [0260] 131 mg (0.5 mmol) 1-[4-(2,4-Difluoro-benzyloxy)-acetophenon and 66 mg (0.5 mmol) 4-allylthiosemicarbazide was refluxed in 1.5 ml ethyl alcohol for 2 days. The mixture was evaporated, then the residue was solidified under diisopropyl ether. The precipitate was filtered, washed with 50% aeous ethyl alcohol, methanol and diisopropyl ether. Thus 98 mg appropriate thiosemicarbazon derivative was obtained. (Yield: 52%).
If necessary, the raw product could be purified by chromatography on TLC plate (eluents: hexane-ethyl acetate=7:3). The pure product was solidified under diisopropyl ether.

| Code | Structure | LCMS | NMR |
|---|---|---|---|
| 29986 | 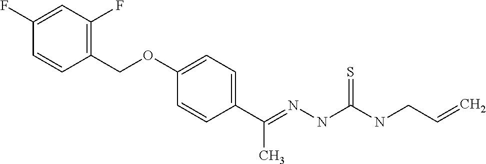 [SMILES: C=CCNC(=S)N/N=C(\C)c1ccc(OCc2ccc(F)cc2F)cc1] | C19H19F2N3OS Mw = 375.44 LCMS purity: 98% m/z 374 [M − H]⁻, 376 [M − H]⁺, Rt. 4.64 min | 10.17 (s, 1H); 8.56 (t, J = 5.8 Hz, 1H); 7.90 (dm, J = 8.8 Hz, 2H); 7.63 (ddd, J = 8.6, 8.6 & 6.7 Hz, 1H); 7.31 (ddd, J = 10.4, 9.5 & 2.6 Hz, 1H); 7.13 (dddd, J = 8.6, 8.6, 2.6 & 0.8 Hz, 1H); 7.04 (dm, J = 8.8 Hz, 2H); 5.92 (ddt, J = 17.2, 10.5 & 5.0 Hz, 1H); 5.16 (s, 2H); 5.14 (ddm, J = 17.2 & 1.5 Hz, 1H); 5.09 (ddm, J = 10.5 & 1.5 |
| | | | Hz, 1H); 4.24 (ddm, J = 5.8 & |
| | | | 5.0 Hz, 2H); 2.28 (s, 3H) |
| 30656 | 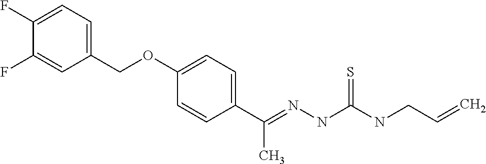 [SMILES: C=CCNC(=S)N/N=C(\C)c1ccc(OCc2ccc(F)c(F)c2)cc1] | C19H19F2N3OS Mw = 375.44 LCMS purity: 96% m/z 374 [M − H]⁻, 376 [M − H]⁺, Rt. 4.70 Min | 10.15 (s, 1H); 8.54 (t, J~5.5 Hz, 1H); 7.89 (dm, J = 8.8 Hz, 2H); 7.54 (ddd, J = 11.5, 8.0 & 2.0 Hz, 1H); 7.46 (ddd, J = 10.7, 8.7 & 8.4 Hz, 1H); 7.32 (dddd, J = 8.4, 4.5, 2.0 & 1.2 Hz, 1H); 7.02 (dm, J = 8.8 Hz, 2H); 5.92 (ddt, J = 17.2, 10.5 & 5.0 Hz, 1H); 5.15 (s, 2H); 5.14 (ddm, J = 17.2 & 1.5 Hz, 1H); 5.09 (ddm, J = 10.5 & |
| | | | 1.5 Hz, 1H); 4.24 (ddm, J = |
| | | | 5.5 & 5.0 Hz, 2H); 2.27 (s, |
| | | | 3H) |
| 30657 | 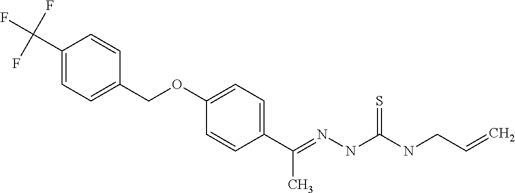 [SMILES: C=CCNC(=S)N/N=C(\C)c1ccc(OCc2ccc(C(F)(F)F)cc2)cc1] | C20H20F3N3OS Mw = 407.46 LCMS purity: 95% m/z 406 [M − H]⁻, 408 [M − H]⁺, Rt. 4.93 min | 10.16 (s, 1H); 8.54 (t, J~5.5 Hz, 1H); 7.89 (dm, J = 8.8 Hz, 2H); 7.76 (dm, J = 8.2 Hz, 2H); 7.68 (dm, J = 8.2 Hz, 2H); 7.03 (dm, J = 8.8 Hz, 2H); 5.92 (ddt, J = 17.2, 10.5 & 5.0 Hz, 1H); 5.29 (s, 2H); 5.14 (ddm, J = 17.2 & 1.5 Hz, 1H); 5.09 (ddm, J = 10.5 & 1.5 Hz, 1H); 4.24 (ddm, J = 5.5 & 5.0 Hz, 2H); 2.27 (s, 3H) |
| 30658 | 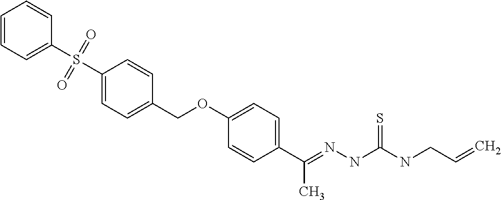 [SMILES: C=CCNC(=S)N/N=C(\C)c1ccc(OCc2ccc(S(=O)(=O)c3ccccc3)cc2)cc1] | C25H25N3O3S2 Mw = 479.62 LCMS purity: 95% m/z 478 [M − H]⁻, 480 [M − H]⁺, Rt. 4.50 min | 10.15 (s, 1H); 8.53 (t, J~5.5 Hz, 1H); 7.98 (dm, J = 7.5 Hz, 2H); 7.96 (dm, J = 7.0 Hz, 2H); 7.87 (dm, J = 8.8 Hz, 2H); 7.70 (tm, J = 7.5 Hz, 1H); 7.68 (dm, J = 7.5 Hz, 2H); 7.62 (ddm, J = 7.5 & 7.0 Hz, 2H); 7.00 (dm, J = 8.8 Hz, 2H); 5.92 (ddt, J = 17.2, 10.5 & 5.0 Hz, 1H); 5.26 (s, 2H); 5.14 (ddm, J = 17.2 & 1.5 Hz, 1H); 5.09 (ddm, J = 10.5 & 1.5 Hz, 1H); 4.23 (ddm, J = |
| | | | 5.5 & 5.0 Hz, 2H); 2.26 (s, |
| | | | 3H) |
| 30659 | 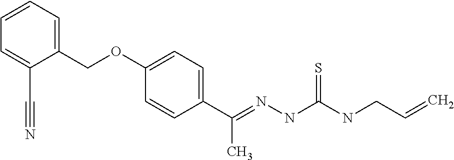 [SMILES: C=CCNC(=S)N/N=C(\C)c1ccc(OCc2ccccc2C#N)cc1] | C20H20N4OS Mw = 364.47 LCMS purity: 95% m/z 363 [M − H]⁻, 365 [M − H]⁺, Rt. 4.32 min | 10.18 (s, 1H); 8.56 (t, J~5.5 Hz, 1H); 7.91 (dm, J = 8.8 Hz, 2H); 7.90 (m, 1H); 7.71- 7.80 (ovl. m, 2H); 7.58 (m, 1H); 7.06 (dm, J = 8.8 Hz, 2H); 5.92 (ddt, J = 17.2, 10.5 & 5.0 Hz, 1H); 5.30 (s, 2H); 5.15 (ddm, J = 17.2 & 1.5 Hz, 1H); 5.09 (ddm, J = 10.5 & 1.5 Hz, 1H); 4.24 (ddm, |
| | | | J = 5.5 & 5.0 Hz, 2H); 2.28 |
| | | | (s, 3H) |

### Preparation of 4-methyl-piperazine-1-carbothioic acid {1-[4-(2,4-difluoro-benzyloxy)-phenyl]-ethylidene}hydrazide of General Formula (IIA)

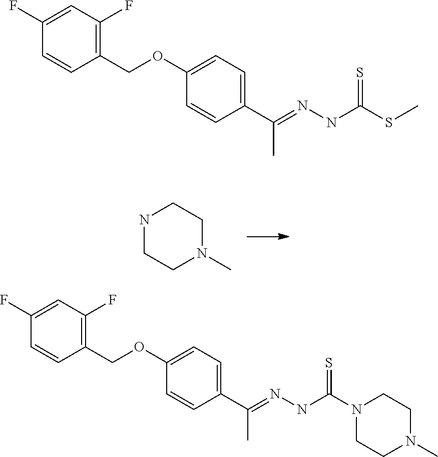 [0263] 121 mg (0.33 mmol) N′-{1-[4-(2,4-difluoro)benzyloxy-phenyl]-ethylidene}-hydrazine-carbodithioic acid methyl ester, 100 mg (1 mmol) 1-methylpiperazine in 6 ml ethyl alcohol was stirred in a microwave reactor at 110 grad Celsius for 1 hour. The mixture was evaporated, then the residue was solidified under diisopropyl ether. The precipitate was filtered, washed with 50% aqueous ethyl alcohol, methanol and diisopropyl ether, then dried on air. Thus 104 mg of title product was obtained. Yield: 75%.
C21H24F2N4OS Mw=418.51
LC/MS purity: 95%, m/z 417 [M−H]−, 419 [M+H]+ Rt. 3.22 min.
1H NMR (300 MHz, DMSO-d6, 1H, ROESY, 13C & ed-HSQC): 10.3 (bs, 1H), 8.46 (d, 1H), 7.48 (s, 1H), 7.37 (s, 1H), 7.36 (t, 1H), 7.08 (d, 1H), 6.97 (d, 1H), 6.37 (d, 1H), 4.18 (t, 2H), 3.92 (s, 3H), 3.66 (s, 3H), 2.45 (m, 2H), 2.38 (bs, 7H), 2.33 (bs, 4H), 2.21 (s, 3H), 2.15 (s, 3H), 1.95 (m, 2H).

### Preparation of thiosemicarbazon Derivatives of General Formula (IIA)

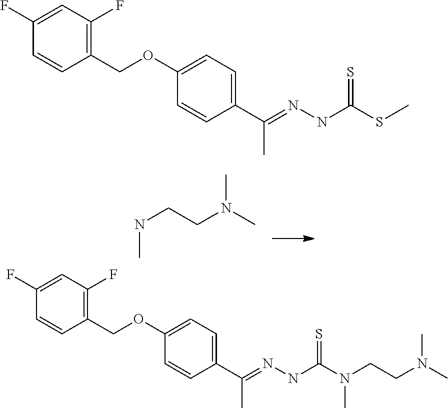 [0268] 121 mg (0.33 mmol) N′-{1-[4-(2,4-difluoro)benzyloxy-phenyl]-ethylidene}-hydrazine-carbodithioic acid methyl ester, 100 mg (1 mmol) N,N,N′-trimethylethylenediamine in 6 ml ethyl alcohol was stirred in a microwave reactor at 110 grad Celsius for 1 hour. The mixture was evaporated, then the residue was solidified under diisopropyl ether. The precipitate was filtered, washed with 50% aqueous ethyl alcohol, methanol and diisopropyl ether, then dried on air. Thus 79 mg of title product was obtained. Yield: 56%.
C21H26F2N4OS Mw=420.53
LC/MS purity: 95%, m/z 419 [M−H]−, 421 [M+H]+ Rt. 3.33 min.
1H NMR (300 MHz, DMSO-d6, 1H, ROESY, 13C & ed-HSQC): 10.87 (br. s, 1H); 7.77 (dm, J=8.8 Hz, 2H); 7.64 (ddd, J=8.6, 8.6 & 6.7 Hz, 1H); 7.30 (ddd, J=10.4, 9.5 & 2.6 Hz, 1H); 7.13 (dddd, J=8.6, 8.6, 2.6 & 0.8 Hz, 1H); 7.08 (br, 2H); 5.16 (s, 2H); 3.74 (m, 2H); 3.28 (s, 3H); 2.55 (m, 2H); 2.25 (br. s, 3H); 2.23 (s, 6H)

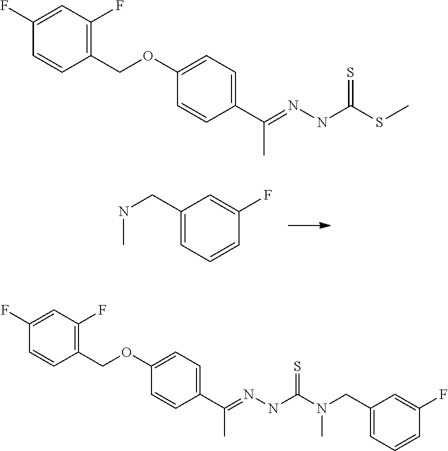

121 mg (0.33 mmol) N′-{1-[4-(2,4-difluoro)benzyloxy-phenyl]-ethylidene}-hydrazine-carbodithioic acid methyl ester, 139 mg (1 mmol) 3-fluoro-N-methylbenzylamine in 6 ml ethyl alcohol was stirred in a microwave reactor at 110 grad Celsius for 1 hour. The mixture was evaporated, then the residue was solidified under diisopropyl ether. The precipitate was filtered, washed with 50% aqueous ethyl alcohol, methanol and diisopropyl ether, then dried on air. Thus 117 mg of title product was obtained. Yield: 77%.
C24H22F3N3OS Mw=457.52
LC/MS purity: 96%, m/z 456 [M−H]−, 458 [M+H]+ Rt. 5.16 min.
1H NMR (300 MHz, DMSO-d6, 1H, ROESY, 13C & ed-HSQC): 9.65 (br. s, 1H); 7.74 (br. 1H); 7.70 (dm, J=8.8 Hz, 2H); 7.64 (ddd, J=8.6, 8.6 & 6.7 Hz, 1H); 7.40 (m, 1H); 7.31 (ddd, J=10.4, 9.5 & 2.6 Hz, 1H); 7.26 (br, 1H); 7.14 (dddd, J=8.6, 8.6, 2.6 & 0.8 Hz, 1H); 7.09 (m, 1H); 7.02 (br, 2H); 5.22 & 5.16 (br, S 2H); 5.15 (s, 2H); 3.27 & 3.21 (br, S 3H); 2.64 & 2.25 (br, S 3H).

### Synthesis of Inhibitors of Mycobacterium Virulence of General Formula II

### Preparation of D23251

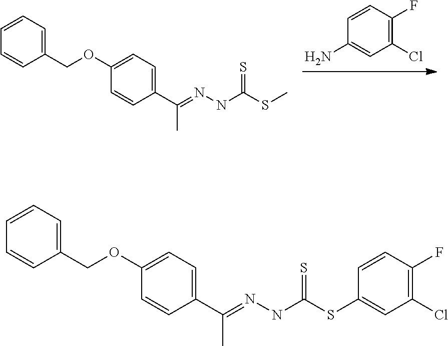 [SMILES: CSC(=S)N/N=C(\C)c1ccc(OCc2ccccc2)cc1 ; Nc1ccc(F)c(Cl)c1 ; C/C(=N\NC(=S)Sc1ccc(F)c(Cl)c1)c1ccc(OCc2ccccc2)cc1] [0277] 330 mg (1 mmol) N′-[1-(4-Benzyloxy-phenyl)-ethylidene]-hydrazinecarbodithioic acid methyl ester and 145 mg (1 mmol) 3-chloro-4-fluoroaniline was stirred in a microwave reactor at 110 grad Celsius for 1 hour. The mixture was evaporated, then it was purified by column chromatography (eluents. hexane-ethyl acetate=7:3). The pure product was solidified under diisopropyl ether. Thus 180 mg of title product was obtained. Yield: 42%.

### Preparation of D23579

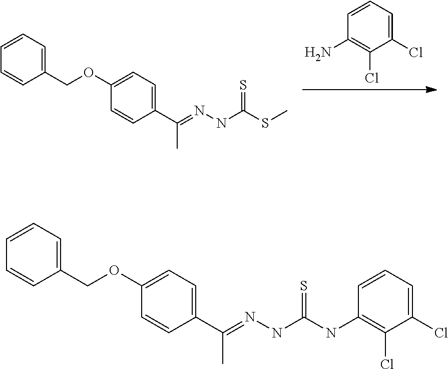 [SMILES: CSC(=S)N/N=C(\C)c1ccc(OCc2ccccc2)cc1 ; Nc1cccc(Cl)c1Cl ; C/C(=N\NC(=S)Nc1cccc(Cl)c1Cl)c1ccc(OCc2ccccc2)cc1] [0279] 330 mg (1 mmol) N′-[1-(4-Benzyloxy-phenyl)-ethylidene]-hydrazinecarbodithioic acid methyl ester and 160 mg (1 mmol) 2,3-dichloroaniline was stirred in a microwave reactor at 110 grad Celsius for 1 hour. The mixture was evaporated, then it was purified by column chromatography (eluents. hexane-ethyl acetate=7:3). The pure product was solidified under diisopropyl ether. Thus 165 mg of title product was obtained. Yield: 37%.

| Code | Structure | LCMS | NMR |
|---|---|---|---|
| 14463 | 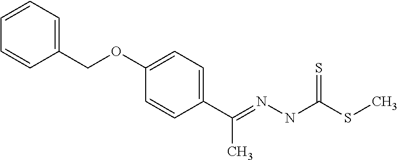 [SMILES: CSC(=S)N/N=C(\C)c1ccc(OCc2ccccc2)cc1] | C17H18N2OS2 Mw = 330.47 LCMS purity: 95% m/z 331 [M − H]⁺ Rt. 3.54 min | 12.37 (s, 1H); 7.82 (dm, J = 8.8 Hz, 2H); 7.46 (dm, J = 7.6 Hz, 2H); 7.40 (ddm, J = 7.6 & 7.4 Hz, 2H); 7.34 (tm, J = 7.4 Hz, 1H); 7.07 (dm, J = 8.8 Hz, 2H); 5.17 (s, 2H); 2.49 (s, 3H); 2.34 (s, 3H) |
| 14466 | 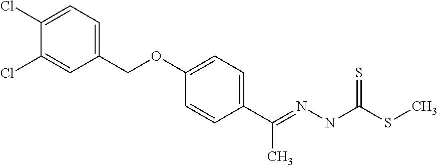 [SMILES: CSC(=S)N/N=C(\C)c1ccc(OCc2ccc(Cl)c(Cl)c2)cc1] | C17H16Cl2N2OS2 Mw = 399.36 LCMS purity: 99% m/z 397 [M − H]⁻, 399 [M − H]⁺, Rt. 3.74 min | 12.39 (s, 1H); 7.83 (dm, J = 8.8 Hz, 2H); 7.74 (d, J = 1.7 Hz, 1H); 7.67 (d, J = 8.2 Hz, 1H); 7.46 (dd, J = 8.2 & 1.7 Hz, 1H); 7.08 (dm, J = 8.8 Hz, 2H); 5.19 (s, 2H); 2.50 (s, 3H); 2.35 (s, 3H) |
| D22670 | 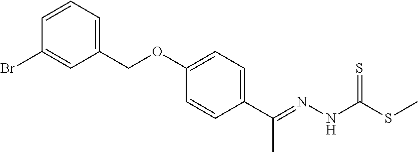 [SMILES: CSC(=S)N/N=C(\C)c1ccc(OCc2cccc(Br)c2)cc1] | C17H17BrN2OS2 Mw = 409.36 LCMS purity: 95% m/z 409 and 411 [M − H]⁺ Rt. 3.66 min | 12.38 (s, 1H); 7.83 (dm, J = 8.8 Hz, 2H); 7.67 (dd, J = 1.5 & 1.5 Hz, 1H); 7.54 (dm, J = 7.8 Hz, 1H); 7.47 (dm, J = 7.5 Hz, 1H); 7.37 (dd, J = 7.8 & 7.5 Hz, 1H); 7.08 (dm, J = 8.8 Hz, 2H); 5.18 (s, 2H); 2.50 (s, 3H); 2.35 (s, 3H) |
| D22663 | 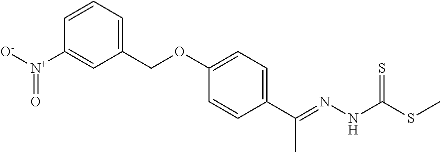 [SMILES: CSC(=S)N/N=C(\C)c1ccc(OCc2cccc([N+](=O)[O-])c2)cc1] | C17H17N3O3S2 Mw = 375.47 LCMS purity: 94% m/z 374 [M − H]⁺, 376 [M − H]⁺ Rt. 3.50 min | 12.36 (s, 1H); 8.33 (dd, J = 1.5 & 1.5 Hz, 1H); 8.21 (dm, J = 8.3 Hz, 1H); 7.93 (dm, J = 7.8 Hz, 1H); 7.84 (dm, J = 8.8 Hz, 2H); 7.71 (dd, J = 8.3 & 7.8 Hz, 1H); 7.11 (dm, J = 8.8 Hz, 2H); 5.34 (s, 2H); 2.49 (s, 3H); 2.35 (s, 3H) |
| D22671 | 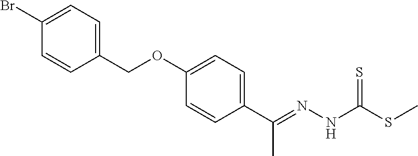 [SMILES: CSC(=S)N/N=C(\C)c1ccc(OCc2ccc(Br)cc2)cc1] | C17H17BrN2OS2 Mw = 409.36 LCMS purity: 95% m/z 409 and 411 [M − H]⁺ Rt. 3.68 min | 12.38 (s, 1H); 7.82 (dm, J = 8.8 Hz, 2H); 7.60 (dm, J = 8.2 Hz, 2H); 7.42 (dm, J = 8.2 Hz, 2H); 7.06 (dm, J = 8.8 Hz, 2H); 5.15 (s, 2H); 2.50 (s, 3H); 2.34 (s, 3H) |
| D23251 | 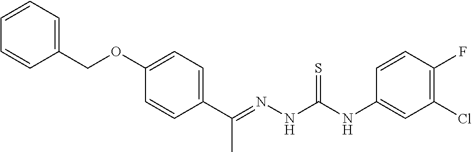 [SMILES: C/C(=N\NC(=S)Nc1ccc(F)c(Cl)c1)c1ccc(OCc2ccccc2)cc1] | C21H24F2N4OS Mw = 427.93 LCMS purity: 94% m/z 426 [M − H]⁻ Rt. 3.56 min | 10.65 (s, 1H); 10.03 (s, 1H); 7.97 (dm, J = 8.8 Hz, 2H); 7.83 (dd, J = 6.8 & 2.5 Hz, 1H); 7.56 (ddd, J = 8.8, 4.4 & 2.5 Hz, 1H); 7.45 (dm, J = 7.6 Hz, 2H); 7.40 (dd, J = 12.0 & 8.8 Hz, 1H); 7.39 (ddm, J = 7.6 & 7.4 Hz, 2H); 7.33 (tm, J = 7.4 Hz, 1H); 7.03 (dm, J = 8.8 Hz, 2H); |
| | | | 5.17 (s, 2H); 2.34 (s, 3H) |
| D22672 | 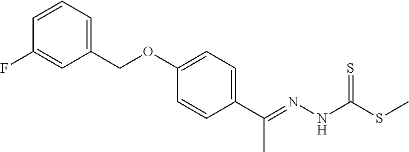 [SMILES: CSC(=S)N/N=C(\C)c1ccc(OCc2cccc(F)c2)cc1] | C17H17FN2OS2 Mw = 348.46 LCMS purity: 99% m/z 349 [M − H]⁺ Rt. 3.55 min | 12.38 (s, 1H); 7.83 (dm, J = 8.8 Hz, 2H); 7.45 (dddm, J = 8.0, 8.0 & 5.7 Hz, 1H); 7.26- 7.33 (ovl. m, 2H); 7.17 (dddm, J = 8.5, 8.0 & 2.5 Hz, 1H); 7.08 (dm, J = 8.8 Hz, 2H); 5.20 (s, 2H); 2.50 (s, 3H); 2.35 (s, 3H) |
| D22668 (BBH7) | 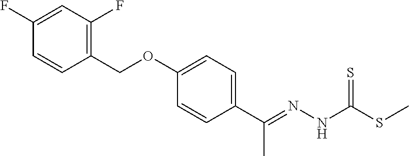 [SMILES: CSC(=S)N/N=C(\C)c1ccc(OCc2ccc(F)cc2F)cc1] | Given in the synthetic route | Given in the synthetic route |
| D23579 | 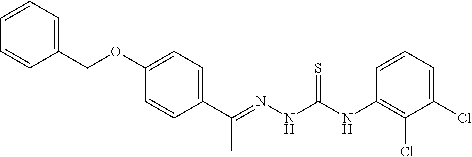 [SMILES: C/C(=N\NC(=S)Nc1cccc(Cl)c1Cl)c1ccc(OCc2ccccc2)cc1] | C22H19Cl2N3OS Mw = 444.39 LCMS purity: 97% m/z 442 and 444 [M − H]⁻ Rt. 3.68 min | 10.83 (s, 1H); 10.12 (s, 1H); 7.96 (dm, J = 8.8 Hz, 2H); 7.79 (dd, J = 8.0 & 1.4 Hz, 1H); 7.56 (dd, J = 8.1 & 1.4 Hz, 1H); 7.46 (dm, J = 7.6 Hz, 2H); 7.40 (dd, J = 8.1 & 8.4 Hz, 1H); 7.39 (ddm, J = 7.6 & 7.4 Hz, 2H); 7.33 (tm, J = 7.4 Hz, 1H); 7.04 (dm, J = 8.8 Hz, 2H); 5.17 (s, |
| | | | 2H); 2.36 (s, 3H) |
| D22647 | 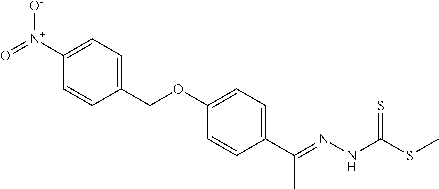 [SMILES: CSC(=S)N/N=C(\C)c1ccc(OCc2ccc([N+](=O)[O-])cc2)cc1] | C17H17N3O3S2 Mw = 375.47 LCMS purity: 97% m/z 374 [M − H]⁻, 376 [M − H]⁺ Rt. 3.53 min | 12.39 (s, 1H); 8.27 (dm, J = 8.6 Hz, 2H); 7.83 (dm, J = 8.8 Hz, 2H); 7.73 (dm, J = 8.6 Hz, 2H); 7.10 (dm, J = 8.8 Hz, 2H); 5.35 (s, 2H); 2.49 (s, 3H); 2.35 (s, 3H) |
| D22646 | 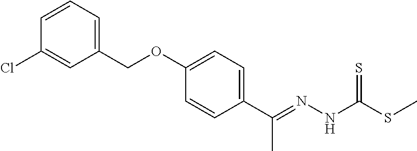 [SMILES: CSC(=S)N/N=C(\C)c1ccc(OCc2cccc(Cl)c2)cc1] | C17H17ClN2OS2 Mw = 364.92 LCMS purity: 97% m/z 365 [M − H]⁺ Rt. 3.65 min | 12.39 (s, 1H); 7.83 (dm, J = 8.8 Hz, 2H); 7.53 (dd, J = 1.5 & 1.5 Hz, 1H); 7.38- 7.46 (ovl. m, 3H); 7.08 (dm, J = 8.8 Hz, 2H); 5.19 (s, 2H); 2.50 (s, 3H); 2.35 (s, 3H) |
| D22648 | 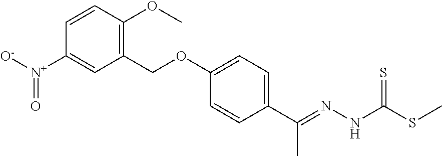 [SMILES: COc1ccc([N+](=O)[O-])cc1COc1ccc(/C(C)=N/NC(=S)SC)cc1] | C18H19N3O4S2 Mw = 405.50 LCMS purity: 97% m/z 404 [M − H]⁻ 406 [M − H]⁺ Rt. 3.56 min | 12.26 (br, 1H); 8.26-8.32 (ovl. m, 2H); 7.84 (dm, J = 8.8 Hz, 2H); 7.31 (dm, J = 9.8 Hz, 1H); 7.11 (dm, J = 8.8 Hz, 2H); 5.19 (s, 2H); 3.99 (s, 3H); 2.50 (s, 3H); 2.35 (s, 3H) |

### Example 2

### Development of a Lung Fibroblast Based HTS for the Identification of Protein Secretion Inhibitors

The screen of small molecule libraries for inhibitors of mycobacterial protein secretion was performed considering the advantage of the cytotoxicity of Mtb for eukaryotic cells upon infection at high multiplicities of infection (MOI).

MRC-5 lung fibroblasts were infected with the wild-type Erdman strain and well-defined attenuated mutants deficient in ESX-1 secretion followed by quantification of metabolical activity in fibroblasts (FIG. 1A). Wild-type Mtb was highly cytotoxic and led to a marked decrease of fluorescence compared to uninfected cells in this fibroblast survival assay (FSA) (FIG. 1B). The ΔRD1 mutant, lacking core-genes in the ESX-1 locus, failed to lyse MRC-5 fibroblasts. Also, infection with a deletion-mutant of the two-component regulatory system PhoPR as well as the ΔespA mutant led to significantly less cytotoxicity due to impaired EsxA secretion (FIG. 1B) (Chen et al., 2013; Gonzalo-Asensio et al., 2008).
Several compounds were tested with known antimycobacterial activity for their ability to protect MRC-5 cells from Mtb-induced cell death. As expected, all compounds with intracellular activity were highly protective whereas aminoglycosides (streptomycin; kanamycin), which fail to penetrate MRC-5 cells, were not (FIG. 1C). To distinguish between anti-virulence compounds and growth inhibitory drugs, all compounds were counter-screened against Mtb in the resazurin reduction microtiter assay (REMA). A putative protein secretion inhibitor was defined as a hit compound that protected fibroblasts from Mtb-induced cell death in the FSA without affecting bacterial growth in the REMA (FIG. 1D).

### Outcome of the Primary and Confirmatory Screens

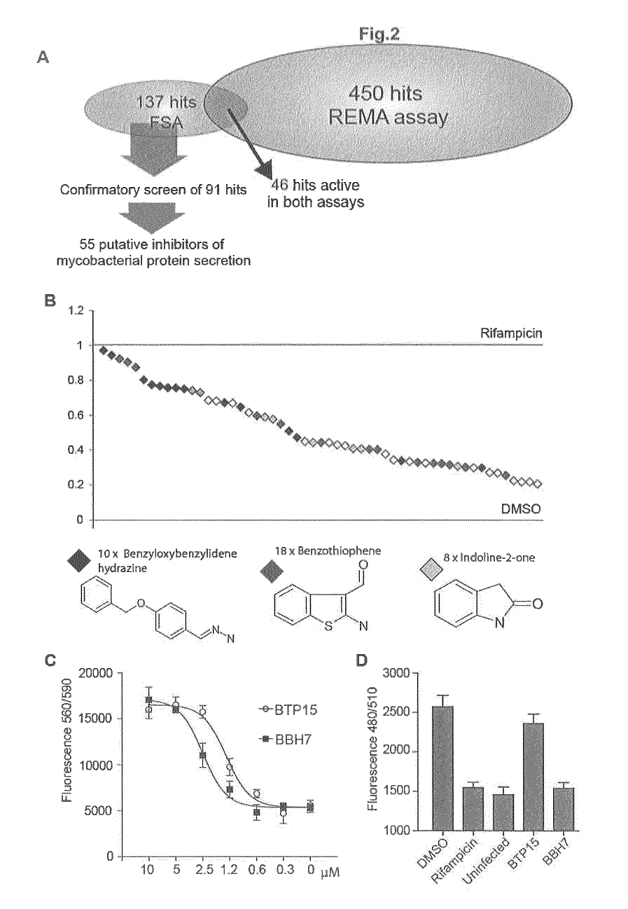

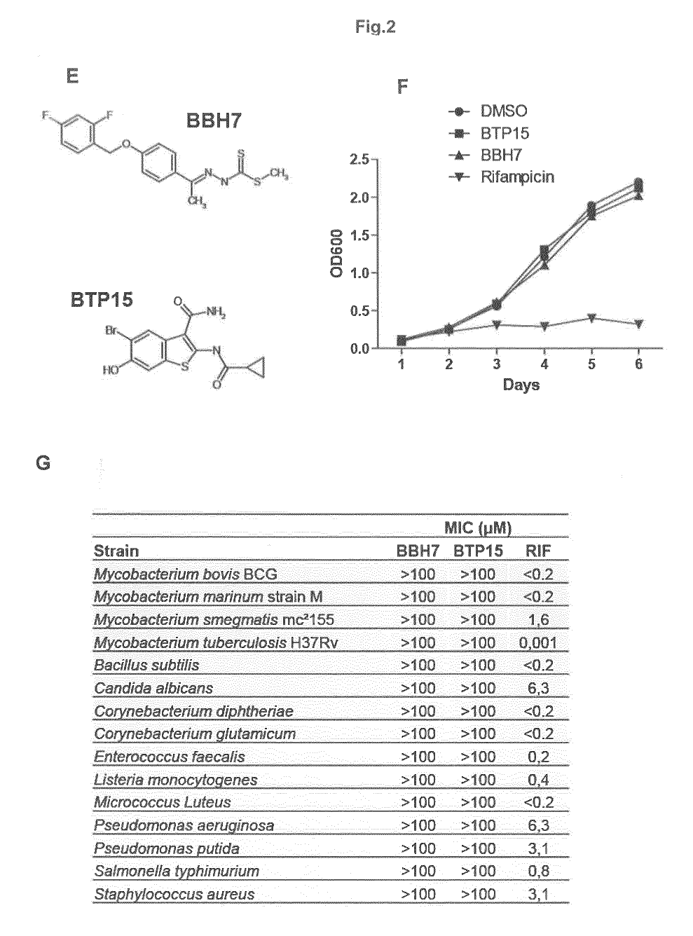

A proprietary library of 10,880 synthetic compounds was screened at a concentration of 5 μM leading to the identification of 450 compounds (hit rate of 4.4%) that inhibited mycobacterial growth in the REMA (FIG. 2A). 137 compounds were protective in the FSA (hit rate of 1.3%), 46 compounds were active in both assays indicating that only 10% of the REMA hit compounds had intracellular activity and were non-cytotoxic for fibroblasts. After a confirmatory screen, 55 of the 91 compounds, which impacted virulence without affecting mycobacterial growth in the primary screen, were validated as true hits (FIG. 2A). Chemo-informatic cluster analysis identified 6 clusters and 9 singletons. FIG. 2B correlates the potency of these hit-compounds to the controls and displays the three most abundant core structures. Of note, several analogs of the benzyloxybenzylidene-hydrazines and the benzothiophenes were almost as efficient as rifampicin in protecting fibroblasts from Mtb-induced cell-death.

For further studies, we selected a benzyloxybenzylidene-hydrazine compound (BBH7) and a benzothiophene compound (BTP15) (FIG. S2A) with particularly good activity in the FSA and a favorable cytotoxicity profile. Both compounds protected fibroblasts in a dose dependent manner (FIG. 2C) with an IC50 of 2.4 μM for BBH7 and 1.2 μM for BTP15, no growth inhibition of Mtb was observed in 7H9 broth at a concentration of 25 μM (FIG. S2B). The Applicant also determined the MIC99 for several other mycobacteria and non-mycobacterial pathogens to be >100 μM for the two compounds (FIG. S2C). Intracellular anti-mycobacterial activity was determined by quantifying Mtb expressing GFP in infected fibroblasts. In this experiment the compounds behaved divergently. BTP15-treated bacteria showed GFP fluorescence comparable to the untreated control whereas no fluorescence was detected in the BBH7 and rifampicin treated samples (FIG. 2D). These data demonstrate that BTP15 had no effect on bacterial viability in the FSA, yet the compound was highly protective for fibroblasts exposed to Mtb whereas BBH7 is a potent inhibitor of intracellular growth.

### Example 3

### BBH7 and BTP15 Inhibit Mycobacterial Protein Secretion at Nanomolar Concentrations

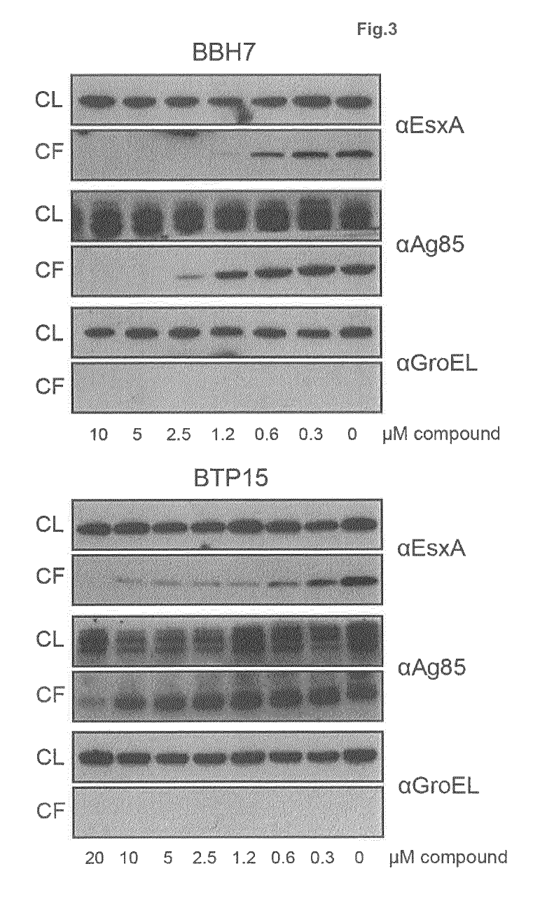

The main aim of the FSA is the identification of potential inhibitors of the ESX-1 secretion system. We exposed Mtb cultures to the compounds, harvested the culture filtrates and quantified EsxA by immunoblot. Intriguingly, both compounds showed dose-dependent secretion inhibition of this major mycobacterium virulence protein (FIG. 3). We also quantified Ag85 complex proteins, which are Tat-secretion dependent substrates. At a concentration of 5 μM BBH7 fully blocked Ag85 secretion. For BTP15 we observed a different pattern as, at concentrations ≦10 μM, Ag85 secretion seemed to be only slightly affected at best. However, 20 μM BTP15 reduced Ag85 secretion and blocked EsxA secretion fully (FIG. 3).

### Example 4

### BTP15 Deregulates Genes Controlled by Two-Component Regulatory Systems

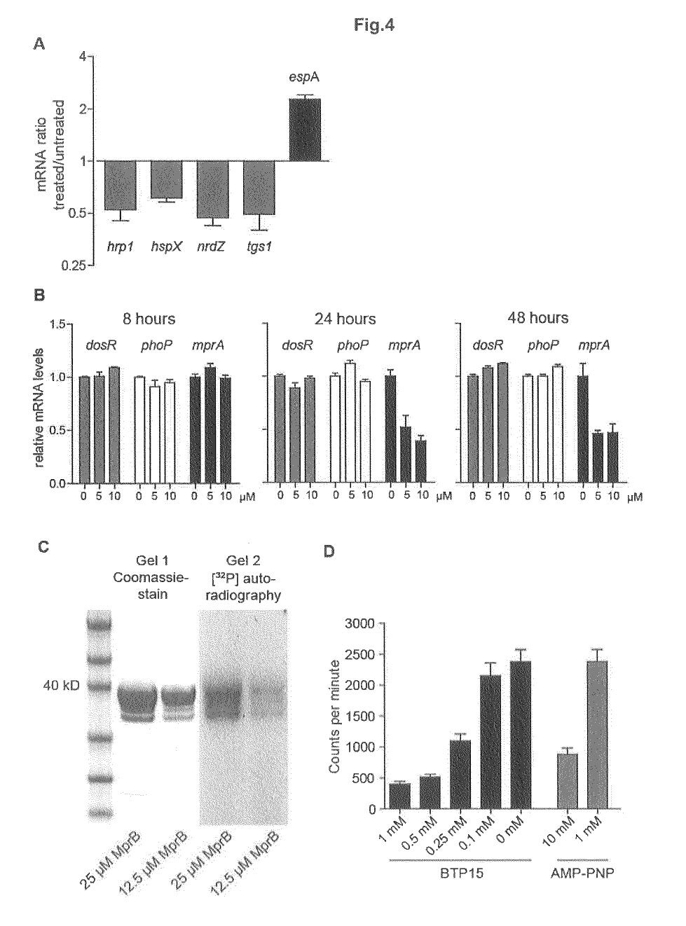

The Applicant performed RNA-seq experiments to gather mechanistic insight from a specific transcriptomic signature of compound-treated Mtb. Only 35 genes were differentially regulated when Mtb was exposed to 5 μM of BTP15 (Table IV). Surprisingly, all 18 genes found to be significantly downregulated were in the DosR (DevR) regulon (Table IV, FIG. 4A). This hypoxia-induced regulon requires the two-component response regulator DosRS which enables the bacteria to enter a “dormant” non-replicative state ensuring intracellular long-term survival and latency (Park et al., 2003).

In Mtb the response regulators PhoPR and MprAB have been shown to link the DosR-regulon and transcriptional regulation of the ESX-1 secretion system via the distal espACD locus (Gonzalo-Asensio et al., 2008; Pang et al., 2013; Pang et al., 2007). Deletion of mprAB leads to upregulation of espA and reduced EsxA secretion (Pang et al., 2013). In the primary RNA-seq experiment espA was upregulated below the threshold of 2 but on analysis by qRT-PCR, espA was among the genes with >2 fold differential regulation (FIG. 4A). Thus, the reduction in EsxA secretion and subsequent loss of virulence observed could be caused by deregulation of the espACD locus. Transcription levels of the regulatory genes dosR, phoP and mprA were then quantified after exposure to BTP15. Interestingly, mprA expression was significantly down-regulated after 24 and 48 hours of drug treatment (FIG. 4B). Since there is considerable overlap among DosR- and MprA-regulated genes, the BTP15 RNA-seq transcript analysis was compared with published gene expression data on mprAB deletion mutants: the majority of the 35 deregulated genes were also differentially regulated in this mutant under different conditions (in grey in Table V) (He et al., 2006; Pang et al., 2007).

### Example 5

### BTP15 is a Kinase Inhibitor that Inhibits MprB Autophosphorylation In Vitro

The Applicant found that treatment of Mtb with BTP15 leads to deregulation of genes controlled by two-component regulatory systems, notably MprAB. It might be that the compound directly affects ATP-dependent signal transducing histidine kinases. Studying histidine phosphorylation is extremely challenging due to the chemical instability of this posttranscriptional modification (Kee and Muir, 2012). An MprB autophosphorylation assay was established using affinity-purified truncated MprB as described (Zahrt et al., 2003). Relatively large amounts of MprB (25 μM) were used in order to detect the MprB phosphohistidine (FIG. 4C), as is common for histidine kinase phosphorylation assays (Saini and Tyagi, 2005).

The Applicant demonstrated dose-dependent inhibition of MprB auto-phosphorylation by BTP15 (FIG. 4D). The non-hydrolyzable ATP analog AMP-PNP can be employed to estimate the potency and specificity of histidine kinase inhibitors having high in vitro IC50 values (Gilmour et al., 2005). When 10 mM AMP-PNP (34× the in vitro IC50 of BTP15) was used only incomplete reduction of the phosphohistidine signal was seen whereas 1 mM AMP-PNP had no effect on auto-phosphorylation (FIG. 4D) indicating that BTP15 is a much stronger inhibitor of MprB auto-phosphorylation than the ATP-analog.

### Example 6

### BBH7 has a Pleiotropic Inhibitory Effect on Mycobacterial Protein Secretion

By immunoblotting, the Applicant found that BBH7 had an impact on two different protein secretion systems at concentrations ≦5 μM (FIG. 3); A 50% reduction of total culture filtrate protein when bacteria were exposed to 5 μM BBH7 was observed. The Applicant further characterized and quantified the secretome of treated and untreated bacteria by LC/MS-MS. These data confirmed the inhibitory effect that BBH7 exerts on the ESX-1 secretion system (Table VI).

In addition, several substrates of the ESX-5 secretion system such as EsxN/EsxM, PE25 and PPE41 were significantly reduced in abundance upon treatment. Reduced secretion of virulence-associated proteins with unknown mechanism of secretion was uncovered showing that BBH7 impacts several independent lines of Mtb pathogenicity

TABLE VI
Secretome analysis of BBH7 treated bacteria
A selection of secreted proteins which were quantified at lower amounts in the culture filtrate of
BBH7 treated bacteria (compound concentration 5 μM). Data are derived from two biological
replicates; proteins were identified by LC-MS/MS as described in the supplementary methods (Table VI).

| | | | | Secretion |
|---|---|---|---|---|
| Id | Name | Product | Function | system |
| Rv0129c | Ag85C | mycolyltransferase | Involved in cell wall synthesis | Tat substrate |
| Rv0164 | TB18.5 | unknown | Predicted outer membrane | unknown |
| | | | protein, essential gene in vitro, | |
| | | | CD8+ and CD4+ T-cell epitope in | |
| | | | mice | |
| Rv1793 | EsxN | unknown | EsxA like proteins | ESX-5 (EsxN) |
| Rv2145c | Wag31 | unknown | Probably involved in cell division | unknown |
| | | | process. Essential gene in vitro | |
| Rv1792 | EsxM | unknown | EsxA like protein | ESX-5 (EsxM) |
| Rv2430c | PPE41 | unknown | PPE family protein, ESX-5 | ESX-5 |
| | | | secretion deficiency leads to | |
| | | | attenuation in vivo and disruption | |
| | | | of cell wall integrity | |
| Rv2431c | PE25 | unknown | PE family protein, ESX-5 | ESX-5 |
| | | | secretion deficiency leads to | |
| | | | attenuation in vivo and disruption | |
| | | | of cell wall integrity | |
| Rv2525c | Rv2525c | unknown | Possible role in biosynthesis of | Predicted Tat |
| | | | the cell wall, deletion results in | substrate |
| | | | enhanced susceptibility to beta- | |
| | | | lactam antibiotics | |
| Rv3208A | TB9.4 | unknown | unknown | unknown |
| Rv3451 | Cut3 | Probable cutinase | Hydrolysis of cutin | Unknown |
| | | precursor | | |
| Rv3682 | PonA2 | penicillin-binding protein, | Required for survival in primary | unknown |
| | | membrane-associated, | murine macrophages | |
| | | transglycosylase and | | |
| | | transpeptidase activities | | |
| Rv3881c | EspB | unknown | Essential for secretion of EsxA | ESX-1 |

### Example 7

### BBH7 Deregulates Several Transmembrane ATPases and Alters Mycobacterial Cell Wall Permeability

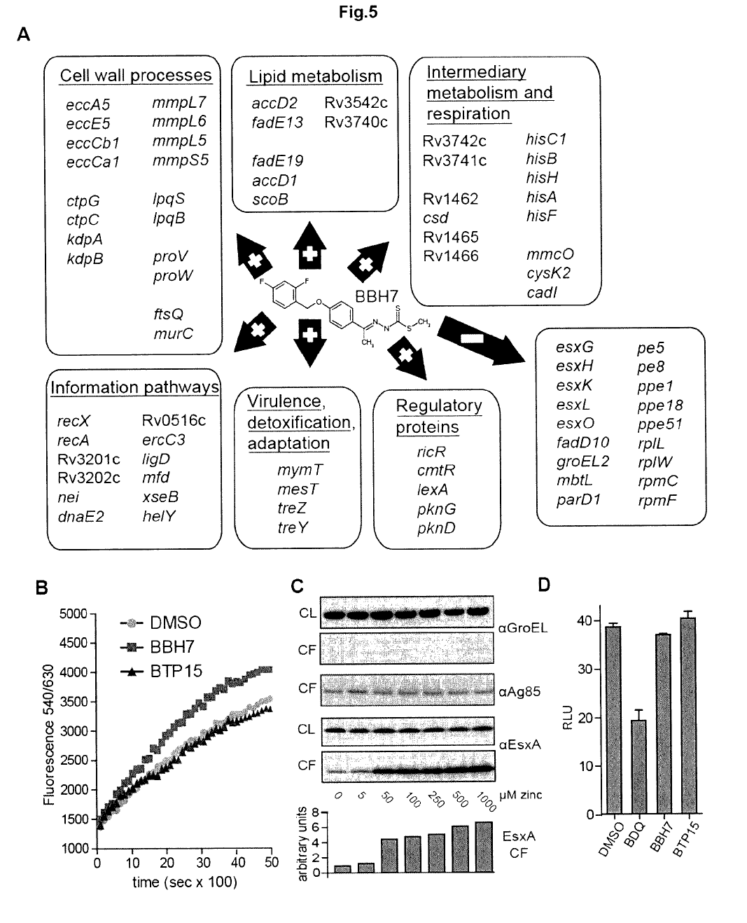

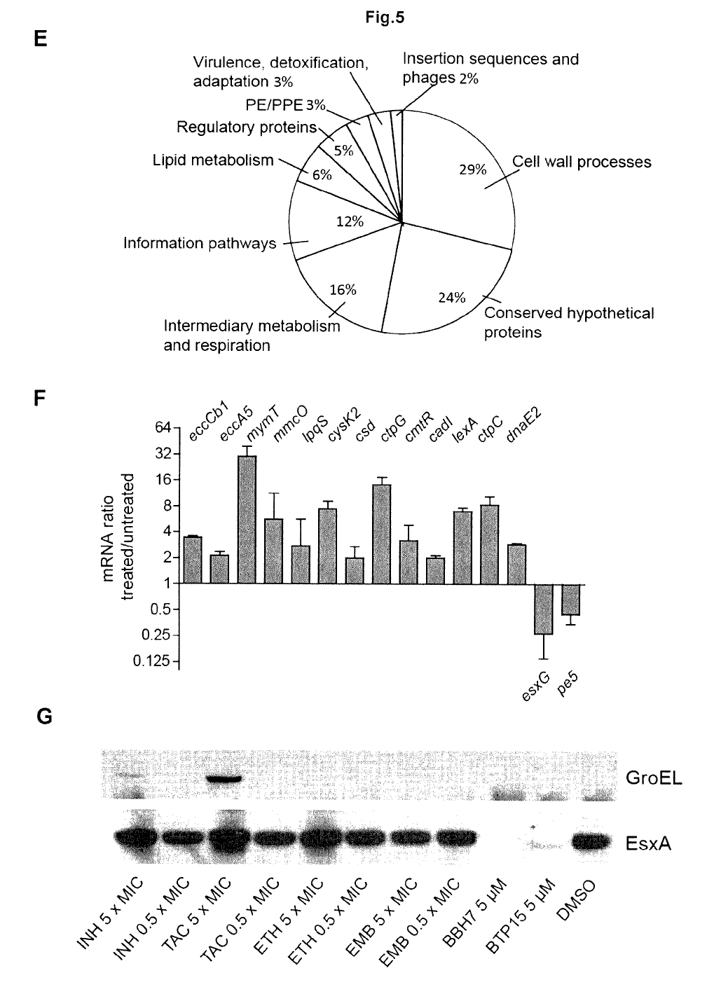

BBH7 had substantial impact on mycobacterial protein secretion. Major changes in the Mtb transcriptome after drug treatment were expected. Indeed, RNA-seq experiments revealed 144 differentially regulated genes (≧2-fold) upon exposure to BBH7. Of these, 121 were upregulated and the gene expression signature mirrors changes primarily associated with cell wall processes and transport (FIG. 5A, FIG. 5E/F). The Applicant found positive regulation for the ESX transmembrane ATPase genes, eccCa1/eccCb1 and eccA5/eccE5, in response to altered ESX-1 and ESX-5 dependent protein secretion. In addition, strong upregulation of the P-type ATPase genes, ctpC and ctpG, indicated disturbed cell membrane/cell wall transport not only for secreted proteins but also for ions such as zinc and copper. There were several other signs for metal-ion overload: strong upregulation of the metallothionein mymT (20 fold), the multicopper oxidase mmcO, the copper-dependent regulator ricR—including the RicR-regulon associated gene lpqS, as well as deregulation of the zinc stress responsive genes cadI, Rv1993, cysK2, esxG and esxH (FIG. 4A, Table VI) (Botella et al., 2011; Maciag et al., 2007). Indirect targets for metal-ion toxicity are Fe—S proteins, explaining the upregulation of the Fe—S cluster biogenesis operon SUF (rv1462-rv1466), and DNA damage leading to a lexA-driven transcriptional response (Rowland and Niederweis, 2012).

To investigate whether BBH7 alters mycobacterial outer membrane permeability, which might explain the transcriptomic pattern associated with metal-ion toxicity, the Applicant performed ethidium bromide (EtBr) uptake assays after treatment with the compounds of interest. These assays identify altered outer membrane permeability by an increase of fluorescence after binding of EtBr to bacterial nucleic acids. It shows that BBH7-treatment increases EtBr accumulation and fluorescence, a sign for perturbed membrane permeability in these bacteria (FIG. 4B). This was not observed with BTP15.

### Example 8

### Zinc Stress Augments EsxA Secretion

The Applicant established that BBH7 alters outer-membrane permeability, leading to signs of zinc and copper stress. Intracellular metal-ion stress might be the link to inhibition of mycobacterial protein secretion upon BBH7 treatment. Thus, Mtb was stressed with physiological concentrations of zinc or copper, as encountered in the phagosome, and determined EsxA secretion levels. Surprisingly, growing cells in media containing elevated levels of ZnSO4 led to a significant and dose-dependent increase of EsxA secretion whereas Ag85 secretion remained unchanged (FIG. 5C). In the presence of 500 μM zinc, a concentration measured in Mtb-infected macrophages (Botella et al., 2011), a sixfold increase in secretion of EsxA was observed (FIG. 5C, lower panel). Elevated concentrations of copper had no effect on EsxA secretion. These findings indicate that BBH7 does not alter mycobacterial protein secretion by zinc or copper intoxication. Furthermore, for the first time, an environmental signal (elevated levels of zinc) that augments EsxA secretion was reported.

Since bacterial transport mechanisms are dependent on the proton motive force which is linked to intracellular ATP-levels, the intracellular ATP concentration was measured after BBH7 treatment. In contrast to treatment with the ATP-synthase inhibitor bedaquiline (BDQ), mycobacterial ATP levels were not found to be reduced by BBH7 (FIG. 5D). To further distinguish BBH7 from well-known, mycobacterial cell wall inhibitors, the Applicant investigated whether such compounds affect EsxA secretion. Isoniazid and ethambutol, as well as the thiourea compounds ethionamide and thiacetazone, had no effect on EsxA secretion at 0.5× MIC (FIG. 5G). At 5× MIC the detection of the cytosolic heat-shock protein GroEL in the culture filtrate indicated cell lysis, which was not observed after BBH7 and BTP15 treatment.

Taken together, these results indicate a novel mechanism of action for BBH7, which alters cell-wall permeability for both export of proteins and import of small molecules, leading to strong upregulation of genes associated with metal ion overload. However, blockage of EsxA secretion by BBH7 does not seem to be caused by zinc/copper intoxication or ATP-depletion.

### Example 9

### BBH7 and BTP15 Promote Phagolysosomal Fusion in Mtb-Infected THP-1 Macrophages Leading to Reduction of the Intracellular Bacterial Load

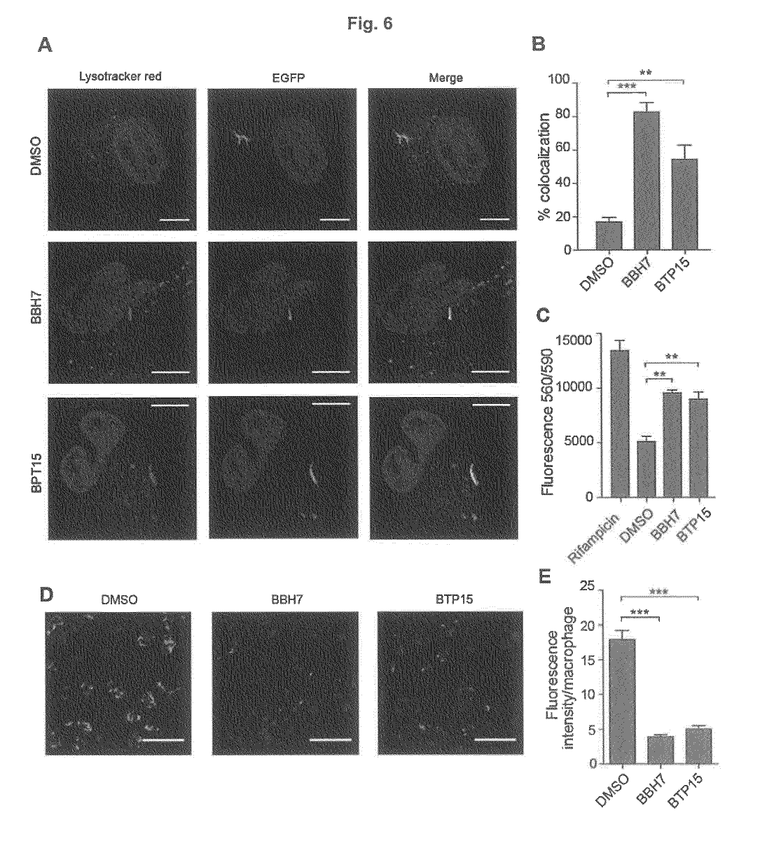

The ESX-1 secretion system plays a decisive role in the arrest of phagosome maturation in Mtb-infected macrophages (MacGurn and Cox, 2007). To test whether BBH7 and BTP15 reverse this phenotype, activated THP-1 macrophages were infected at low MOI with Mtb cells expressing GFP and treated for 7 days. Subsequently, acidic compartments were stained with Lysotracker Red and co-localization of the dye with fluorescent mycobacteria quantified by confocal microscopy. Treated bacteria were found in acidic compartments at a significantly higher rate than untreated bacteria (FIGS. 6A and B). In a second experiment, activated THP-1 macrophages were infected at a higher MOI and then, quantified surviving macrophages as well as intracellular fluorescent mycobacteria. Treatment with both BBH7 and BTP15 protected THP-1 cells from Mtb-induced cell death (FIG. 6C) and greatly reduced the intracellular bacterial load (FIG. 6D/E).

### Example 10

### ESX-1 Inhibitory Activity of Inhibitors of Mycobacterium Virulence

TABLE VII

| Benzothiophene | | |
|---|---|---|
| name | Activity | Structure |
| D38979 | 60% | 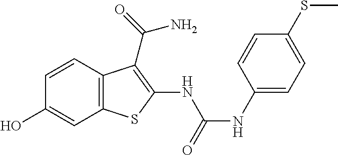 [SMILES: CSc1ccc(NC(=O)Nc2sc3cc(O)ccc3c2C(N)=O)cc1] |
| D39317 | 33% | 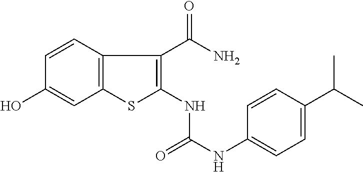 [SMILES: CC(C)c1ccc(NC(=O)Nc2sc3cc(O)ccc3c2C(N)=O)cc1] |
| D39322 | 40% | 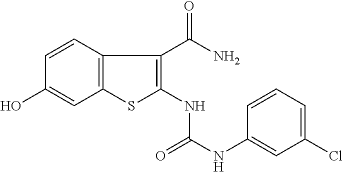 [SMILES: NC(=O)c1c(NC(=O)Nc2cccc(Cl)c2)sc2cc(O)ccc12] |
| D45756 | 32% | 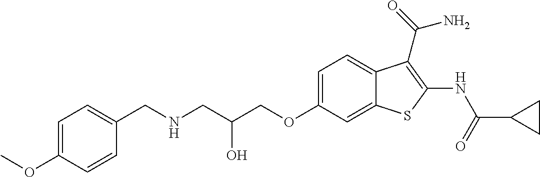 [SMILES: COc1ccc(CNCC(O)COc2ccc3c(C(N)=O)c(NC(=O)C4CC4)sc3c2)cc1] |
| D49399 | 33% | 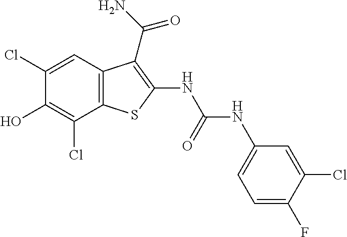 [SMILES: NC(=O)c1c(NC(=O)Nc2ccc(F)c(Cl)c2)sc2c(Cl)c(O)c(Cl)cc12] |
| D51275 | 41% | 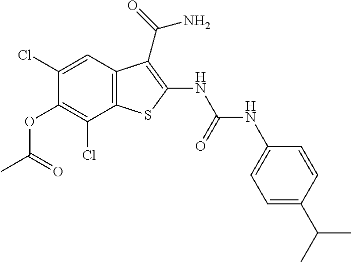 [SMILES: CC(=O)Oc1c(Cl)cc2c(C(N)=O)c(NC(=O)Nc3ccc(C(C)C)cc3)sc2c1Cl] |
| D58298 | 33% | 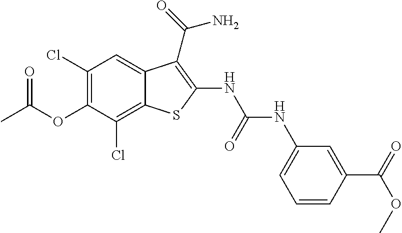 [SMILES: COC(=O)c1cccc(NC(=O)Nc2sc3c(Cl)c(OC(C)=O)c(Cl)cc3c2C(N)=O)c1] |
| D58845 | 87% | 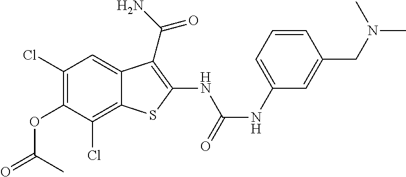 [SMILES: CC(=O)Oc1c(Cl)cc2c(C(N)=O)c(NC(=O)Nc3cccc(CN(C)C)c3)sc2c1Cl] |
| D60166 | 55% | 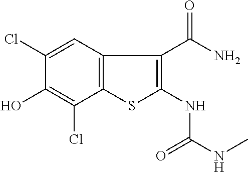 [SMILES: CNC(=O)Nc1sc2c(Cl)c(O)c(Cl)cc2c1C(N)=O] |
| D63134 | 26% | 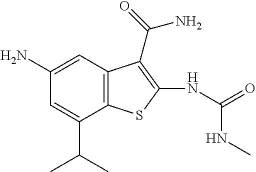 [SMILES: CNC(=O)Nc1sc2c(C(C)C)cc(N)cc2c1C(N)=O] |
| D70865 | 77% | 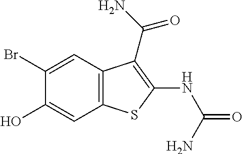 [SMILES: NC(=O)Nc1sc2cc(O)c(Br)cc2c1C(N)=O] |
| D70866 | 80% | 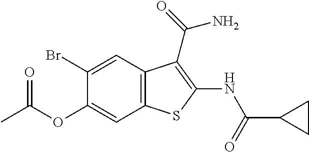 [SMILES: CC(=O)Oc1cc2sc(NC(=O)C3CC3)c(C(N)=O)c2cc1Br] |
| D71014 | 90% | 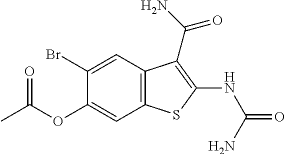 [SMILES: CC(=O)Oc1cc2sc(NC(N)=O)c(C(N)=O)c2cc1Br] |
| D71103 (BTP15) | 92% | 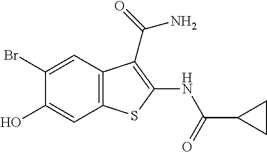 [SMILES: NC(=O)c1c(NC(=O)C2CC2)sc2cc(O)c(Br)cc12] |
| D39321 | 45% | 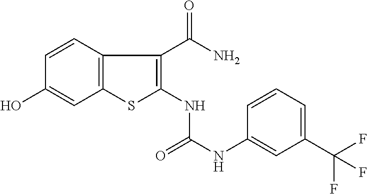 [SMILES: NC(=O)c1c(NC(=O)Nc2cccc(C(F)(F)F)c2)sc2cc(O)ccc12] |
| D22670 | 67% | 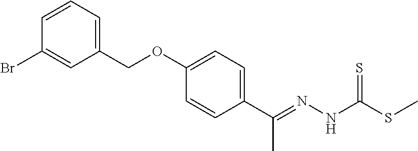 [SMILES: CSC(=S)N/N=C(\C)c1ccc(OCc2cccc(Br)c2)cc1] |
| D22663 | 47% | 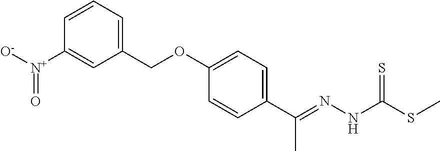 [SMILES: CSC(=S)N/N=C(\C)c1ccc(OCc2cccc([N+](=O)[O-])c2)cc1] |
| D22671 | 51% | 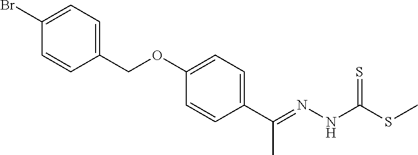 [SMILES: CSC(=S)N/N=C(\C)c1ccc(OCc2ccc(Br)cc2)cc1] |
| D23251 | 34% | 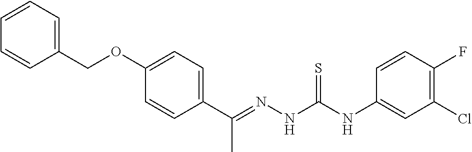 [SMILES: C/C(=N\NC(=S)Nc1ccc(F)c(Cl)c1)c1ccc(OCc2ccccc2)cc1] |
| D22672 | 94% | 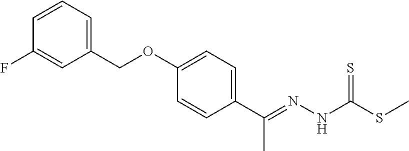 [SMILES: CSC(=S)N/N=C(\C)c1ccc(OCc2cccc(F)c2)cc1] |
| D22668 (BBH7) | 97% | 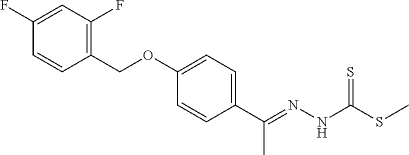 [SMILES: CSC(=S)N/N=C(\C)c1ccc(OCc2ccc(F)cc2F)cc1] |
| D23579 | 75% | 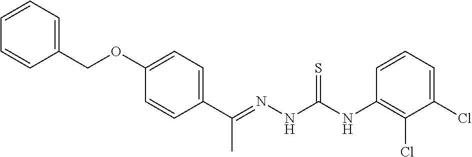 [SMILES: C/C(=N\NC(=S)Nc1cccc(Cl)c1Cl)c1ccc(OCc2ccccc2)cc1] |
| D22647 | 30% | 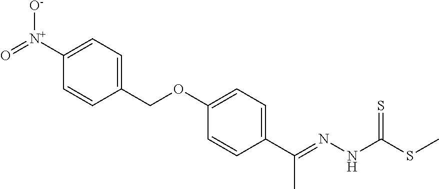 [SMILES: CSC(=S)N/N=C(\C)c1ccc(OCc2ccc([N+](=O)[O-])cc2)cc1] |
| D22646 | 76% | 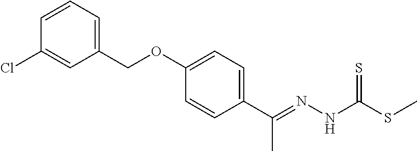 [SMILES: CSC(=S)N/N=C(\C)c1ccc(OCc2cccc(Cl)c2)cc1] |
| D22648 | 65% | 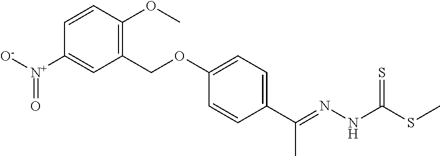 [SMILES: COc1ccc([N+](=O)[O-])cc1COc1ccc(/C(C)=N/NC(=S)SC)cc1] |

1. An inhibitor of mycobacterium virulence of general formula (I) 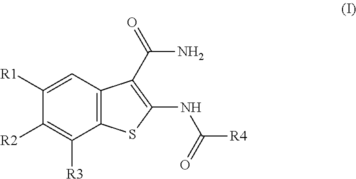
wherein:
R1 is selected from the group consisting of H, halogen, amine;
R2 is selected from the group consisting of H, —OH, substituted alkoxy, —O(CH₂)ₙ—NH₂ with n=2 to 5, acyloxy;
R3 is selected from the group consisting of H, halogen, C₁-C₆ alkyl;
R4 is selected from the group consisting of amine, substituted amine, C₃-C₈ cycloalkyl, substituted benzene,
or general formula (IIA) 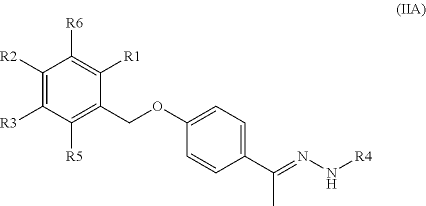
wherein:
R1 is selected from the group consisting of H, halogen, alkoxy;
R2 is selected from the group consisting of H, halogen, nitrogen dioxide, —CF₃, —CO—ORₐ wherein Rₐ is C₁-C₆ alkyl, —SO₂—R_{b} wherein R_{b} is phenyl;
R3 is selected from the group consisting of H, halogen, nitrogen dioxide;
R4 is selected from the group consisting of H, —C(S)—S—R′, —C(S)—NH—R′, —C(S)—NR_{c}—R′ wherein R′ is H, C₁-C₆ alkyl, C₁-C₆ alkene or substituted benzene and R_{c} is substituted C₁-C₆ alkyl;
R5 is selected from the group consisting of H, halogen, cyano group;
R6 is selected from the group consisting of H, halogen,
or, general formula (III) 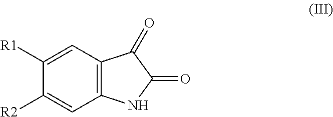
wherein:
R1 is selected from the group consisting of H, halogen, nitrogen dioxide, carboxy, alkoxy, heteroaryl;
R2 is selected from the group consisting of H, halogen;
R3 is selected from the group consisting of C₁-C₆ alkyl heteroaryl, ═N—NH—R″ wherein R″ is substituted aryl;
and/or pharmaceutically acceptable salts thereof.

2. The inhibitor of mycobacterium virulence of general formula (I), according to claim 1, wherein R4 is cyclopropane.

3. The inhibitor of mycobacterium virulence of general formula (I), according to claim 1 or 2, of formula

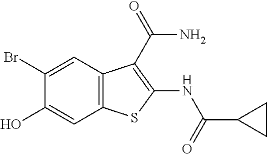

4. The inhibitor of mycobacterium virulence of general formula (I), according to claim 1, selected from the group comprising:

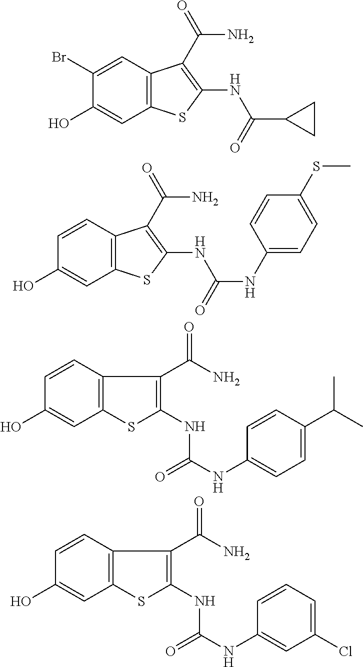

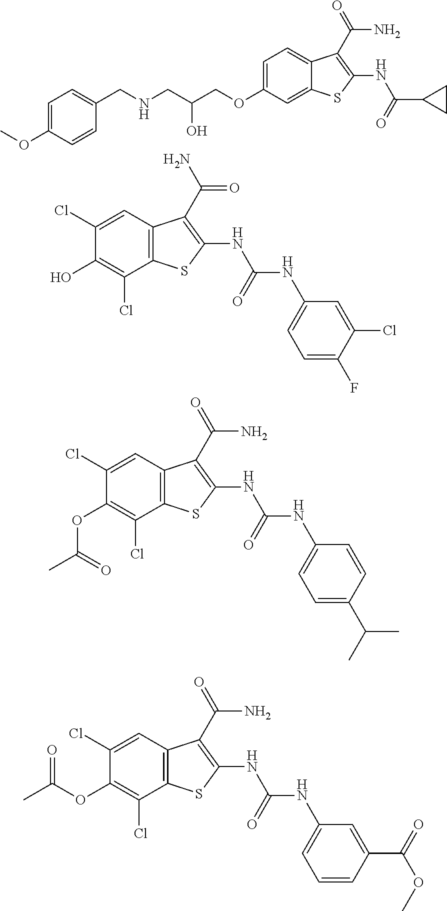

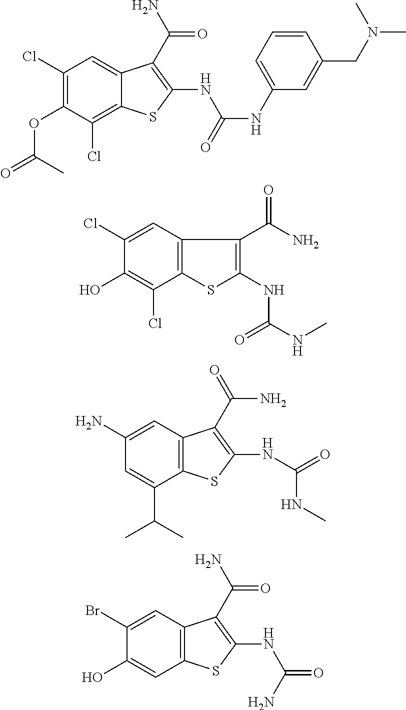

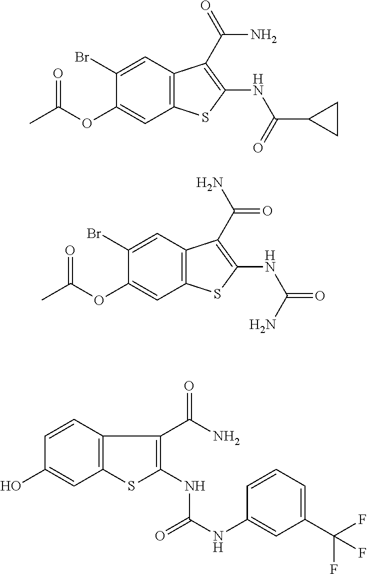

5. The inhibitor of mycobacterium virulence of general formula (IIA), according to claim 1, wherein R4 represents —C(═S)—S—CH3.

6. The inhibitor of mycobacterium virulence of general formula (IIA), according to claim 1 or 5, of formula

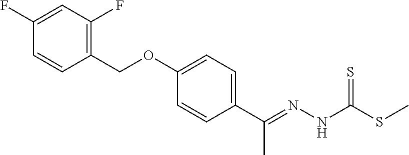

7. The inhibitor of mycobacterium virulence of general formula (IIA), according to claim 1, selected from the group comprising:

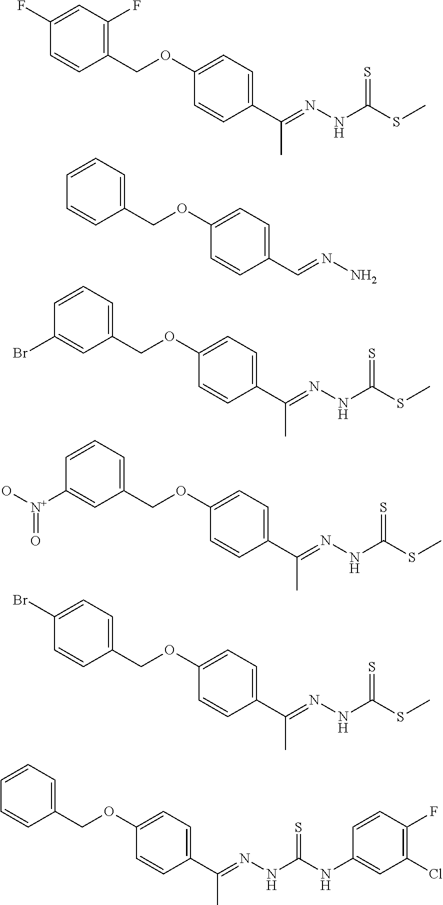

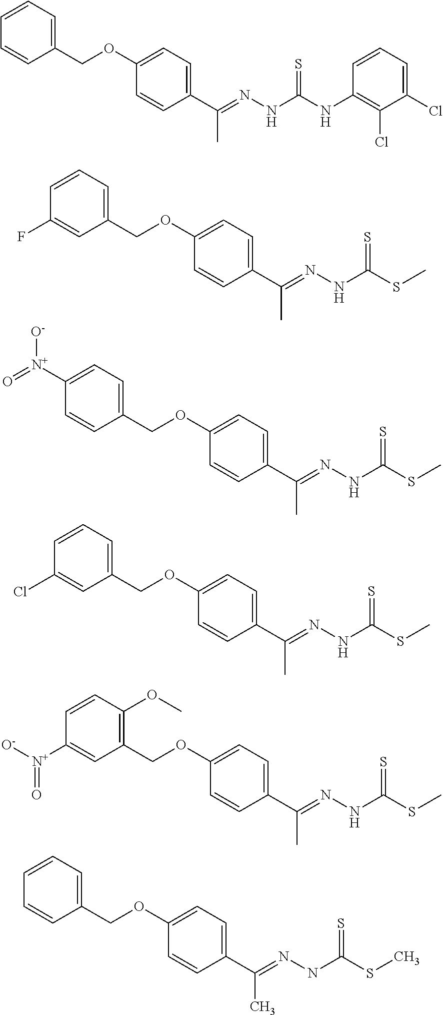

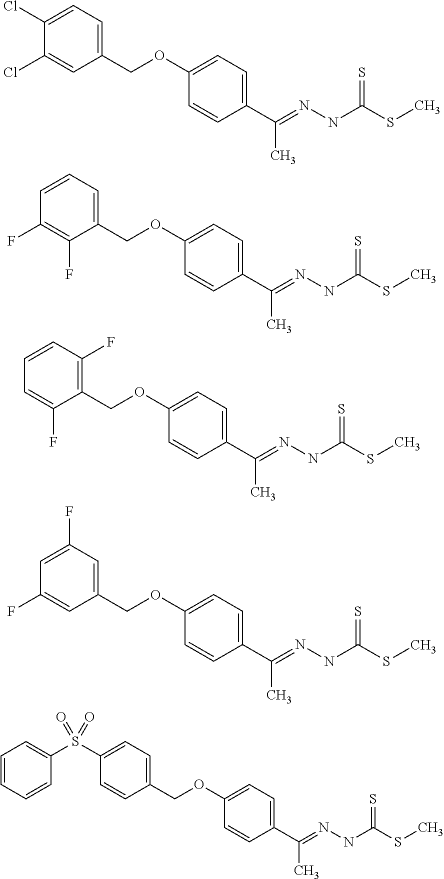

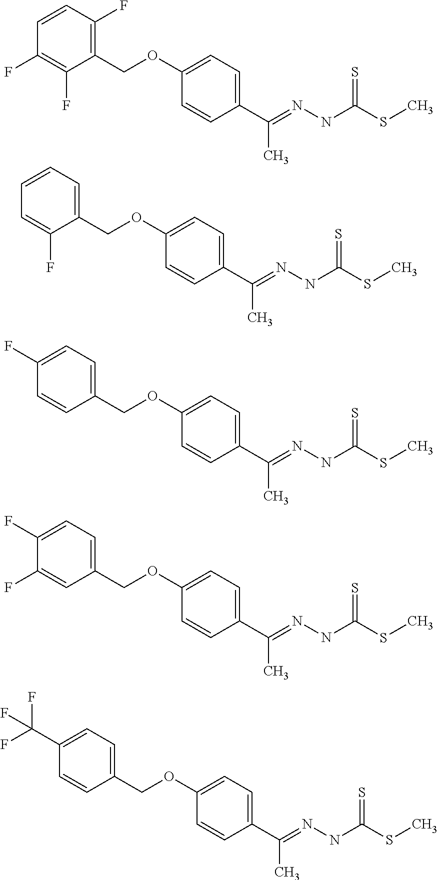

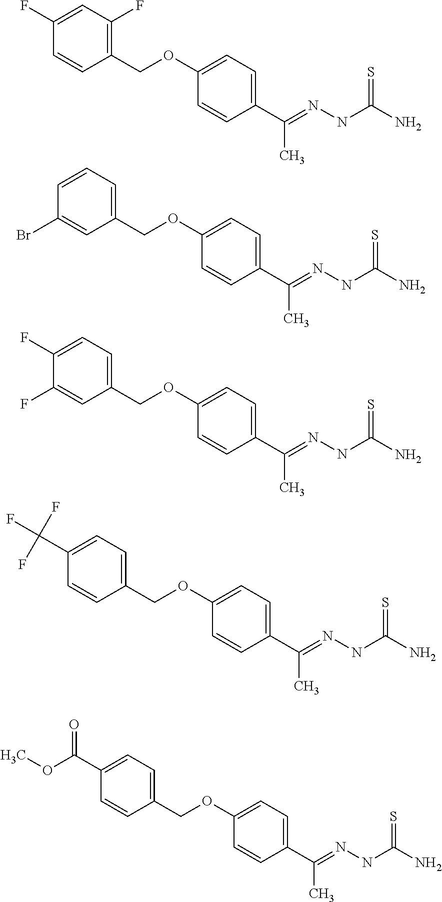

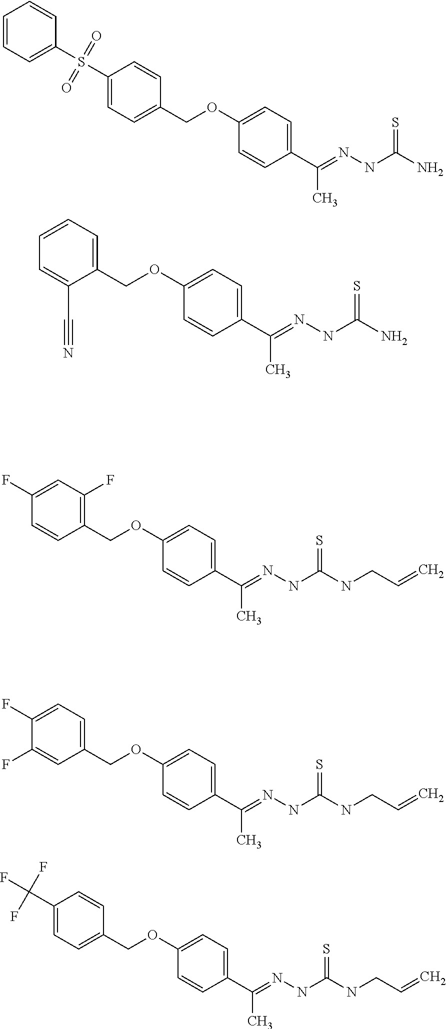

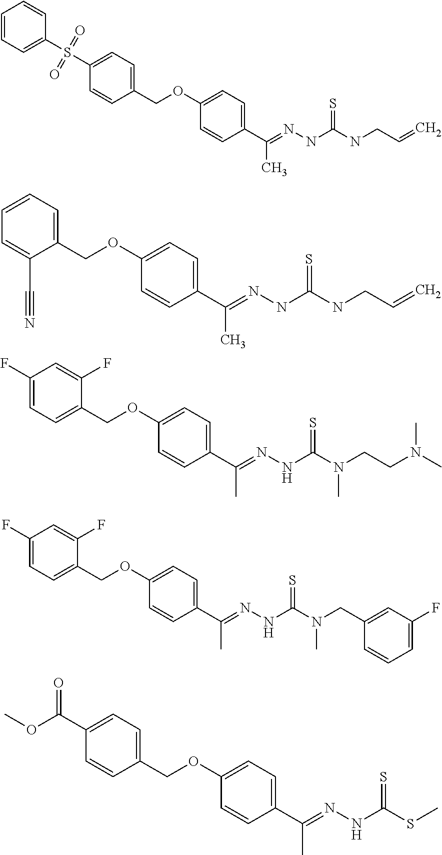

8. The inhibitor of mycobacterium virulence of general formula (III), according to claim 1, wherein R3 represents

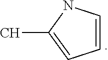

9. The inhibitor of mycobacterium virulence of general formula (III), according to claim 1, selected from the group comprising:

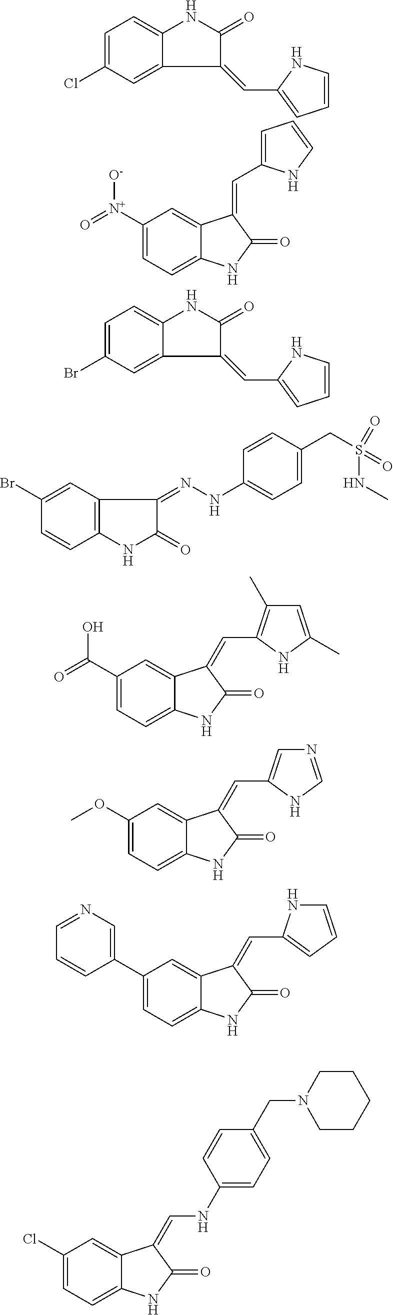

10. The inhibitor of mycobacterium virulence according to any of the preceding claims, for use as a medicament.

11. The inhibitor of mycobacterium virulence according to any of the preceding claims, for use in the treatment and/or prevention of tuberculosis.

12. A pharmaceutical composition comprising the inhibitor of mycobacterium virulence according to any of the preceding claims and a pharmaceutically acceptable carrier, diluent or excipient.

13. A screening method for identifying inhibitors of mycobacterium virulence, said method comprising
a) infecting eukaryotic cells and/or macrophages with wild-type Mtb-Erdman strain at high multiplicities of infection (MOI),
b) contacting said infected eukaryotic cells and/or infected macrophages with an inhibitor to be screened,
c) quantifying metabolic activity in said eukaryotic cells and/or macrophages,
wherein said inhibitor fulfills the following criteria:
i) protects said eukaryotic cells and/or macrophages from Mycobacterium tuberculosis (Mtb)-induced cell death during and after the exposure to said inhibitor,
ii) does not influence Mtb growth, and
iii) either inhibits the histidine kinase MprB in Mtb or affects metal ion homeostasis in Mtb.

14. The screening method of claim 13, wherein the influence of inhibitors of mycobacterium virulence on Mtb growth is verified against Mtb in the resazurin reduction microtiter assay (REMA).
